SEQUENCE LISTING (4 sequences)

SEQ ID NO: 1 - protein, 13 aa, Artificial
  N-terminal region of a peptide able to interact with the mammal glucagon-like peptide-1 receptor
  VARIANT (2)..(2): X is an amino acid selected from alanine and glycine
  VARIANT (10)..(10): X is an amino acid selected from valine and leucine
  VARIANT (12)..(12): X is an amino acid selected from serine and lysine
  VARIANT (13)..(13): X is an amino acid selected from tyrosine and glutamine
HXEGTFTSDXSXX

SEQ ID NO: 2 - protein, 30 aa, Artificial Sequence
  peptide
  MOD_RES (30)..(30): AMIDATION
HAEGTFTSDVSSYLEGQAAKEFIAWLVKGR

SEQ ID NO: 3 - protein, 31 aa, Artificial Sequence
  peptide
HAEGTFTSDVSSYLEGQAAKEFIAWLVKGRG

SEQ ID NO: 4 - protein, 13 aa, Artificial Sequence
  peptide
HAEGTFTSDVSSY

(19) United States
(12) Patent Application Publication
(10) Pub. No.: US 2023/0079395 A1
(43) Pub. Date: Mar. 16, 2023

(54) OPHTHALMIC PHARMACEUTICAL COMPOSITION AND USE THEREOF

(71) Applicant: FERRER INTERNACIONAL, S.A., SANT CUGAT DEL VALLÈS (ES)

(72) Inventors: Carmen LAGUNAS ARNAL, BARCELONA (ES); Andrés FERNÁNDEZ GARCÍA, BARCELONA (ES); Laurence LACHAMP, CASTELLDEFELS (ES); Roland CHÉRIF-CHEIKH, CASTELLDEFELS (ES); Fréderic LACOMBE, CASTELLDEFELS (ES)

(73) Assignee: FERRER INTERNACIONAL, S.A., SANT CUGAT DEL VALLÈS (ES)

(21) Appl. No.: 17/795,836

(22) PCT Filed: Feb. 12, 2021
(86) PCT No.: PCT/EP2021/053476

§ 371 Date: Jul. 27, 2022

(30) Foreign Application Priority Data
Feb. 13, 2020 (EP) 20382101.2

(51) Int. Cl. A61K 38/26; A61K 9/00; A61K 9/08; A61K 9/19; A61P 27/02

(52) U.S. Cl. CPC A61K 38/26; A61K 9/0048; A61K 9/08; A61K 9/19; A61P 27/02

(57) ABSTRACT

The present invention relates to the field of pharmaceutical compositions for ocular diseases, in particular, retinal neurogenerative diseases. The invention provides pharmaceutical compositions to be applied topically in the eyes, including peptides and methods for preparing them thereof. This invention further relates to ophthalmic pharmaceutical composition for use in the topical eye treatment and/or prevention of a retinal neurodegenerative disease.

This application claims the benefit of European Patent Application EP20382101.2 filed Feb. 13, 2020.

### TECHNICAL FIELD

The present invention relates to the field of pharmaceutical compositions for ocular diseases, in particular, retinal neurogenerative diseases. The invention provides pharmaceutical compositions to be applied topically in the eyes, including peptides and methods for preparing them thereof. This invention further relates to ophthalmic pharmaceutical composition for use in the topical eye treatment and/or prevention of a retinal neurodegenerative disease.

### BACKGROUND ART

Retinal neurodegenerative diseases refer to retinal conditions characterized by progressive neuronal loss. Diabetic retinopathy, age-related macular degeneration, glaucoma and retinitis pigmentosa are considered retinal diseases in which neurodegeneration plays an essential role.

An in depth analysis of these diseases, their critical sites, as well as of possible ways of protection and ways leading to recovery can be extracted from Schmidt et al., “Neurodegenerative Diseases of the Retina and Potential for the Protection and Recovery”, Current Neuropharmacology—2008, Vol. No. 6, pp.: 164-178.

Diabetic retinopathy (DR) is the most common complication of diabetes and remains the leading cause of blindness among working-age individuals in developed countries. Current treatments for DR such as laser photocoagulation, intravitreal injections of corticosteroids or anti-VEGF agents are indicated in too advanced stages of the disease and are associated with significant adverse effects. In addition, all these treatments are quite expensive, exhibit a reduced benefit/risk ratio, require a vitreoretinal specialist and most of them are invasive. Consequently, new treatments for treating early stages of the disease are urgently needed.

Diabetic retinopathy (DR) has been classically considered to be a microcirculatory disease of the retina. However, during last years an increasing amount of evidence clearly suggest that retinal neurodegeneration is an early event in the pathogenesis of DR which participates in the microcirculatory abnormalities that occur in DR as can be deduced from Simó et al. (“Neurodegeneration is an early event in diabetic retinopathy: therapeutic implications”, Br. J. Ophthalmol., 2012, vol. 96, pp. 1285-1290) on behalf of the European Consortium for Early Treatment of Diabetic Retinopathy (EUROCONDOR).

In the case of DR the neurodegeneration (loss of effective neurons) starts at the early stages of the disease and produces functional abnormalities such as the loss of both chromatic discrimination and contrast sensitivity. These alterations can be detected by means of specific electrophysiological studies in diabetic patients even with less than two years of diabetes duration, that is, before microvascular lesions can be detected under ophthalmologic examination. In addition, a delayed multifocal ERG (electroretinography) implicit time (mfERG-IT) detects early microvascular abnormalities. Furthermore, neuroretinal degeneration initiates and/or activates several metabolic and signalling pathways which will participate in the microangiopathic process, as well as in the disruption of the blood-retinal barrier (a crucial element in the pathogenesis of DR).

The early stages of retinal neurodegenerative diseases or neurodegeneration associated with these pathologies are not currently treated, although they would prevent advanced lesions, such as microcirculatory problems leading to retinal neovascularization. Thus, at early stages, in particular of DR, no treatment is applied, and the standard follow-up of the patients is conducted.

Diabetes is a group of chronic diseases characterized by hyperglycemia. To prevent diabetic complications, it is essential to reduce hyperglycemia using systemic blood glucose lowering agents. Therefore, any glucose lowering drug could be theoretically beneficial to prevent or arrest diabetic complications, included DR. However, there is a lack of information regarding a direct effect of antidiabetic agents on DR independently of their action in reducing blood glucose levels. By way of example, the glucagon-like peptide 1 agonists known as exenatide (Byetta, Amylin Pharmaceuticals) and liraglutide (Victoza, Novo Nordisk) are used for treating type 2 diabetes by promoting the lowering of blood glucose levels. Moreover, it is known that these agonists give rise to an improvement in the associated diseases of metabolic syndrome such as obesity and high blood pressure. Also, the patent application WO2007062434 discloses a pharmaceutical composition to be intranasally administered, in which the same glucagon-like peptide 1 (GLP-1) is delivered for treating metabolic syndrome and diabetic complications, including DR.

From the above, therefore, it is known that administration of such glucagon-like peptide 1 agonists also improves or attenuates DR symptoms, since the leading cause or the origin of the disease, in particular the high levels of glucose in blood, is at final instance improved. Nonetheless, these treatments are not deprived of systemic adverse effects. If, moreover, these substances have to reach the retina at therapeutic concentrations, crossing through the so-called blood-retinal barrier, high doses are required thus increasing the adverse effects.

In developed countries, diabetic patients are included in well-defined treatment protocols derived from widely-adopted guidelines, however, in spite of such systemic and effective blood glucose-lowering treatments, the fact is that more than 30% of diabetic population develops DR, suggesting that standard systemic control is not enough to efficiently prevent DR development.

At present, there are no specific treatments for retinal neurodegenerative diseases. In the particular case of DR, this means that there are no specific treatments to protect the neuroretina from damage (leading to loss of neurons), particularly, for the background retinopathy or non-proliferative DR. Therefore, new pharmacological treatments for the disease, and especially at early stages when neurodegeneration seems to be starting are needed. Early treatment of DR will be effective in reducing the progression to advanced stages needing aggressive therapies such as laser photocoagulation or intravitreal injections .

WO2014131815 discloses peptides with a sequence length from 13 to 50 amino acids, the N-terminal region of said peptides consisting in the sequence HXaa¹EGTFTSDXaa²SXaa³Xaa⁴ (SEQ ID NO: 1) wherein: Xaa¹ is an amino acid selected from alanine and glycine; Xaa² is an amino acid selected from valine and leucine; Xaa³ is an amino acid selected from serine and lysine; Xaa⁴ is an amino acid selected from tyrosine and glutamine; and histidine is the N-terminal residue; for use in the topical treatment and/or prevention of retinal neurodegenerative diseases, in particular diabetic retinopathy. It is disclosed that the glucagon-like peptide 1 receptor (GLP-1R) was present in human retina, and contrary to all previous assumptions, they were able demonstrate that substances of peptide nature with a molecular weight ranging from 3.35 kDa to 4.18 kDa could reach the retina when applied topically to eyes (i.e. in the cornea). Thus, the topical use (topical eye use) of peptides comprising from 13 to 50 amino acids and including SEQ ID NO: 1, which sequence is considered responsible of the activation of the GLP-1R and is also present in the mammal GLP-1 is proposed.

However, to this date there has been no practical ophthalmic pharmaceutical composition sufficiently stable for administering peptides comprising from 13 to 50 amino acids and including SEQ ID NO: 1 such as GLP-1(7-36)NH₂ and other peptide incretins that is safe, can achieve a high bioavailability, and is suitable for frequent delivery of the drug. One problem with this approach is that, of various known biologically active polypeptides, certain peptides, including GLP-1(7-36)NH₂, that have an isoelectric point (pl, hereinafter) in an acidic or neutral pH range tend to become unstable in an acidic or neutral solution.

For example, observations by the present inventors have revealed that for several solutions prepared of GLP-1(7-36)NH₂ the peptide becomes insoluble when the solution is stored over several days. Thus, preparing a stable solution with the peptide solubilized and suitable for use for its topical eye application needs to be developed.

Therefore, it is substantially challenging to find a stable liquid composition to administer these peptides in the form of solution preparation. An ophthalmic drug delivery system's goal is to achieve a therapeutic concentration of the active drug in the target tissue for an appropriate duration.

It is thus an object of the present invention to provide ophthalmic pharmaceutical compositions which are stable and well tolerated for use in the treatment of retinal neurodegenerative diseases and, more specifically, for use in the treatment of diabetic retinopathy, age-related macular degeneration, glaucoma and retinitis pigmentosa.

A problem still exists in the art to provide topical ophthalmic preparations that contain low concentrations of GLP-1 but retain stability and efficacy for periods of time that translate into an acceptable shelf life for the composition. The invention provides topical ophthalmic compositions comprising peptides that, when applied topically in the eye (i.e. in the cornea or conjunctival fornix), are able to reach the retina, despite their high molecular weight, and achieve effective concentrations for abrogating the evolution of retinal neurodegenerative diseases.

### SUMMARY OF THE INVENTION

The inventors have found topical ophthalmic preparations that contain low concentrations of peptides showing pharmaceutical stability that translates into an acceptable shelf life for the composition. These topical ophthalmic preparations enable more convenient topical administration (topical eye administration) of peptides comprising from 13 to 50 amino acids and including SEQ ID NO: 1, which sequence is considered responsible of the activation of the GLP-1R and is also present in the mammal GLP-1.

Thus, in a first aspect, the present invention relates to an ophthalmic pharmaceutical composition comprising a peptide or a pharmaceutically acceptable salt or solvate thereof with a sequence length from 13 to 50 amino acids, the N-terminal region of said peptide consisting in the sequence:
HXaa¹EGTFTSDXaa²SXaa³Xaa⁴(SEQ ID NO: 1) wherein:
   Xaa¹ is an amino acid selected from alanine and glycine;
   Xaa² is an amino acid selected from valine and leucine;
   Xaa³ is an amino acid selected from serine and lysine;
   Xaa⁴ is an amino acid selected from tyrosine and glutamine; and
histidine is the N-terminal residue; and
   one or more pharmaceutically acceptable excipients or carriers;

wherein the pH value of the composition is between 4.0 and 4.8 and the osmolality ranges between 0.5 and 200 mOsm/kg.

GLP-1 (glucagon-like peptide-1) is an endogenous insulinotropic peptide that is secreted from the L cells of the gastrointestinal tract in response to food (“incretin response”). GLP-1 by acting thorough its receptor (GLP-1R), shows potent effects on glucose-dependent insulin secretion, insulin gene expression, islet beta-cell neogenesis, gastrointestinal motility, energy homeostasis and food intake. The GLP-1 receptor (GLP-1R) is a member of the peptide hormone binding class B1 (secretin-like receptors) family of seven transmembrane spanning, heterotrimeric G-protein coupled receptors (GPCRs). GLP-1Rs have a broad distribution and they are found in the pancreas, adipose tissue, muscle, heart, the gastrointestinal tract and the liver. In addition, GLP-1Rs are found throughout the central nervous system (i.e. hypothalamus, striatum, brain stem, substantia nigra, subventricular zone and also retina), and there is some evidence that GLP-1R stimulation by GLP-1 exerts neuroprotective effects in both the central and peripheral nervous systems.

Human GLP-1 is a 30 or 31 amino acid residue peptide originating from preproglucagon which is produced and secreted by intestinal enteroendocrine L-cells such as in the distal ileum, in the pancreas and in the brain. The human preproglucagon is identified with the UniProt database Accession Number P01275, Feb. 6, 2007; Version 3. The processing of preproglucagon to give GLP-1 (7-36)amide, GLP-1 (7-37) and GLP-2 occurs mainly in the L-cells. A simple system is used to describe fragments and analogues of this peptide. Thus, for example, Gly⁸-GLP-1(7-37) designates a fragment (analogue) of GLP-1 formally derived from GLP-1 by deleting the amino acid residues Nos. 1 to 6 and substituting the naturally occurring amino acid residue in position 8 (Ala) by Gly. Similarly, Lys³⁴(N^{ε}-tetradecanoyl)-GLP-1 (7-37) designates GLP-1 (7-37) wherein the ε-amino group of the Lys residue in position 34 has been tetradecanoylated.

The designation GLP-1(1-36) indicates that the peptide fragment in question comprises the amino acid residues from (and including) number 1 to (and including) number 36 when counted from the N-terminal end of the parent peptide, GLP-1. Similarly, the designation GLP-1(7-37) designates that the fragment in question comprises the amino acid residues from (and including) number 7 to (and including) number 37 when counted from the N-terminal end of the parent peptide, GLP-1. The amino acid sequence of GLP-1(7-36)amide (SEQ ID NO: 2) corresponds to:

His-Ala-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Gly-Arg

wherein the C-terminal end is —CONH₂; whereas the amino acid sequence of GLP-1(7-37) (SEQ ID NO: 3) corresponds to:

His-Ala-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Gly-Arg-Gly

These peptides have been reported to reach the retina and exert therein a neuroprotection effect, providing the therapeutic advantages of providing a local action to the eye and minimising the associated systemic adverse effects.

As it is shown below, the combination of an acidic pH, comprised in the range from 4 to 4.8, together with an osmolality comprised from 0.5 to 200 mOsm/kg, provides a long-term stability (up to 12 months) of the peptide formulated in the ophthalmic composition.

The topical treatment and/or prevention is a topical eye treatment and/or prevention, thus in the eye surface (i.e. in the cornea or conjunctival fornix), since the peptides can reach the retina when applied topically to eyes. This applies to any of the embodiments and combination of embodiments disclosed in the present invention.

In a second aspect, the present invention relates to a lyophilizate comprising the peptide as defined in the first aspect of the invention or a pharmaceutically acceptable salt thereof, and a pharmaceutically acceptable amount of a stabilizing agent or buffering agent, wherein said lyophilizate is suitable for preparing the ophthalmic pharmaceutical composition according to any one of the preceding claims by reconstitution.

This second aspect can be alternatively be formulated as a lyophilizate obtainable by lyophilization of a solution comprising:
a) a pharmaceutically effective amount of the peptide as defined in the first aspect and/or a pharmaceutically acceptable salt thereof,
b) a pharmaceutically acceptable amount of stabilizing agent or buffering agent, and
c) water
   wherein said lyophilizate is suitable for preparing the ophthalmic pharmaceutical compositions according to any one of the preceding claims by reconstitution.

In a third aspect, the present invention relates to a process for preparing the ophthalmic pharmaceutical composition of the first aspect, which comprises the step of reconstituting the lyophilizate as defined in the second aspect of the invention, with an aqueous vehicle composition comprising one or more pharmaceutically acceptable carriers or excipients, particularly an aqueous vehicle composition comprising at least one viscosifying agent and optionally at least one preservative.

The invention also provides a process for preparing the ophthalmic pharmaceutical composition of the first aspect, which comprises:
a) providing a lyophilizate comprising a pharmaceutically effective amount of the peptide as defined in the first aspect and/or a pharmaceutically acceptable salt thereof and a pharmaceutically acceptable amount of stabilizing agent or buffering agent,
b) providing a vehicle composition comprising at least one viscosifying agent and optionally at least one preservative; and
c) reconstituting the lyophilizate of step a) with the vehicle composition of step b) to form an ophthalmic pharmaceutical composition.

In a fourth aspect, the present invention relates to an ophthalmic pharmaceutical composition obtainable by the process of the third aspect.

In a fifth aspect, the present invention relates to a kit comprising the lyophilizate as defined in the second aspect of the invention, and a physiologically acceptable vehicle composition comprising one or more pharmaceutically acceptable excipients or carriers, for reconstituting the peptide.

The components of the formulation can be included in the kit in the form of a mixed powder or fluid. In the kit of the present invention, all components can be included in the mixed solution, or a part thereof can be included in the mixed solution and partly in powder form. In one embodiment, the physiologically acceptable vehicle comprises at least one viscosifying agent and optionally at least one preservative.

In a sixth aspect, the present invention provides a kit comprising the ophthalmic pharmaceutical composition of the first and fourth aspect, a container for holding the pharmaceutical composition and a drop dispenser adapted for administering a volume, for example, about 10 to 100 μl volume of the composition per drop, preferably about 10 to 50 μl volume, more preferably about 20 to 40 μl volume.

In a final aspect, the present invention relates to an ophthalmic pharmaceutical composition of the first or fourth aspect of the invention for use in the topical eye treatment and/or prevention of a retinal neurodegenerative disease. This aspect can be alternatively be formulated as the use of the ophthalmic pharmaceutical composition of the first or fourth aspect of the invention in the manufacture of a medicament for the treatment and/or prevention of a retinal neurodegenerative disease. This aspect can be alternatively be formulated as a method for the treatment and/or prevention of a retinal neurodegenerative disease, the method comprising administering a therapeutically effective amount of the ophthalmic pharmaceutical composition of the first or fourth aspect of the invention to a subject in need thereof.

### DETAILED DESCRIPTION OF THE INVENTION

For the sake of understanding, the following definitions are included.

The expression “neuroprotection in the early stages of diabetic retinopathy” relates to any treatment or prophylactic method carried out before advanced stages of DR (proliferative DR (PDR)) are established. For “early stages of diabetic retinopathy” is to be understood as the time in which, due to the presence of diabetes, functional and microvascular abnormalities can be detected in the eye (i.e. chromatic discrimination, contrast sensitivity and electroretinography abnormalities), but the characteristic neovascularization of PDR has not yet been fully established.

“Human glucagon like peptide-1 (7-36)amide (GLP-1 (7-36)amide)”, and “human glucagon like peptide-1 (7-37) (GLP-1 (7-37)” relate to the fragments derived from human proglucagon and comprising from amino acid 7 to 36 or from amino acid 7 to 37, respectively, of the amino acid sequence of said human proglucagon.

As “analogue of human GLP-1 (7-37)” is to be understood a peptide wherein one or more amino acid residues of the GLP-1 (7-37) have been substituted by another amino acid residue and/or wherein one or more of the amino acid residues of the GLP-1 (7-37) have been deleted and/or wherein one or more amino acid residues have been added to the GLP-1 (7-37).

The expression “therapeutically effective amount” as used herein, refers to the amount of a compound (i.e., the peptide) that, when administered, is sufficient to prevent development of, or alleviate to some extent, one or more of the symptoms of the disease which is addressed. The particular dose of compound administered according to this invention will of course be determined by the particular circumstances surrounding the case, including the compound administered, the route of administration, the particular condition being treated, and the similar considerations.

The term “pharmaceutically acceptable” as used herein pertains to compounds, materials, compositions, and/or dosage forms which are, within the scope of sound medical judgment, suitable for use in contact with the tissues of a subject (e.g. human) without significant toxicity, irritation, allergic response, or other problem or complication, commensurate with a reasonable benefit/risk ratio. Each carrier, excipient, etc., must also be “acceptable” in the sense of being compatible with the other ingredients of the pharmaceutical composition. It must also be suitable for use in contact with the tissue or organ of humans and animals without excessive toxicity, irritation, allergic response, immunogenicity or other problems or complications commensurate with a reasonable benefit/risk ratio. Suitable carriers, excipients, etc. can be found in standard pharmaceutical texts, and include, as a way of example preservatives, agglutinants, humectants, emollients, and antioxidants.

Used herein, the term “pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, phosphate, acetate, trifluoroacetate, citrate, tosylate, maleate, and oxalate.

The expression “excipients and/or carriers” refers to acceptable materials, compositions or vehicles. Each component must be pharmaceutically acceptable in the sense of being compatible with the other ingredients of the composition. It must also be suitable for use in contact with the tissue or organ of humans and non-human animals without excessive toxicity, irritation, allergic response, immunogenicity or other problems or complications commensurate with a reasonable benefit/risk ratio. Examples of suitable acceptable excipients are solvents, dispersion media, diluents, or other liquid vehicles, dispersion or suspension aids, surface active agents, isotonic agents, thickening or emulsifying agents, preservatives, solid binders, lubricants and the like. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

The skilled artisan will appreciate that pharmaceutically acceptable salts of compounds may be prepared. These pharmaceutically acceptable salts may be prepared in situ during the final isolation and purification of the compound, or by separately reacting the purified compound in its free acid or free base form with a suitable base or acid, respectively.

The compound of the invention may be in the form of a salt, e.g. a pharmaceutically acceptable salt or a solvate, e.g. a hydrate.

“Solvate” or “solvates” of a compound refer to those compounds, as defined above, which are bound to a stoichiometric or non-stoichiometric amount of a solvent. In certain embodiments, solvents are volatile, non-toxic, and/or acceptable for administration to humans in trace amounts, and/or water.

The “N-terminal region” or “the N-terminus” (also known as the amino-terminus, NH₂-terminus, N-terminal end or amine-terminus, all of them used herewith as interchangeable expressions) refers to the start of a protein or polypeptide terminated by an amino acid with a free amine group (—NH₂). The convention for writing peptide sequences is to put the N-terminus on the left and write the sequence from N- to C-terminus. When the protein is translated from messenger RNA, it is created from N-terminus to C-terminus. For “N-terminal residue” is to be understood the residue in a peptide that has an amino group that is free, or at least not acylated by another amino-acid residue (it may, for example, be acylated or formylated), is called N-terminal; it is at the N-terminus. The residue that has a free carboxyl group, or at least does not acylate another amino-acid residue, (it may, for example, acylate ammonia to give —NH—CHR—CO—NH₂), is called C-terminal.

For the purposes of the present invention, a pharmaceutical composition comprising an active substance is considered to be “stable” if said ingredient degrades less or more slowly than it does on its own and/or in known pharmaceutical compositions.

As above exposed, the inventors propose for the first time an ophthalmic pharmaceutical composition of GLP-1 and analogues for retinal neurodegenerative diseases (retinal diseases in which neurodegeneration plays an essential role) that, in addition of being non-aggressive, is useful in the treatment of the early stages of these diseases, and in particular in the treatment of DR.

Ocular administration of drugs is primarily associated with the need to treat ophthalmic diseases. Eye surface is the most easily accessible site for topical administration of a medication. Ophthalmic preparations are sterile products, suitably compounded and packaged for instillation into the eye. They are easily administered by the nurse or the patient himself, they have quick absorption and effect, less visual and systemic side effects, increased shelf life and better patient compliance.

In a particular embodiment of the present invention, the ophthalmic pharmaceutical composition according to the first aspect has a sequence length from 30 to 50 amino acids.

For the purposes of the present invention, any ranges given include both the lower and the upper end-points of the range.

Unless otherwise stated, the one or more of the amino acids forming the peptides of the invention can have L- or D-configuration.

Another particular embodiment is an ophthalmic pharmaceutical composition comprising a peptide or a pharmaceutically acceptable salt or solvate thereof with a sequence length from 30 to 40 amino acids, the N-terminal region of said peptide consisting of the sequence:
HXaa¹EGTFTSDXaa²SXaa³Xaa⁴(SEQ ID NO: 1) wherein:
   Xaa¹ is an amino acid selected from alanine and glycine;
   Xaa² is an amino acid selected from valine and leucine;
   Xaa³ is an amino acid selected from serine and lysine;
   Xaa⁴ is an amino acid selected from tyrosine and glutamine; and
histidine is the N-terminal residue; and

one or more pharmaceutically acceptable excipients or carriers; wherein the pH value of the composition is between 4.0 and 4.8 and the osmolality ranges between 0.5 and 200 mOsm/kg.

Yet in another particular embodiment, the ophthalmic pharmaceutical composition according to the first aspect has a sequence length from 13 to 40 amino acids.

In a preferred embodiment of the pharmaceutical composition as herein disclosed, the pharmaceutically acceptable salt of the peptide is selected from acetate, hemitartrate and hydrochloride; preferably the pharmaceutically acceptable salt of the peptide is an acetate.

The compound of the invention refers to a therapeutically active compound, as well as any prodrugs thereof and pharmaceutically acceptable salts, hydrates and solvates of the compound and the prodrugs.

In another embodiment of the present invention, the peptides are those comprising at the N-terminal region of the amino acid sequence consisting in SEQ ID NO: 1 in which Xaa¹ is alanine, Xaa² is valine, Xaa³ is serine, and Xaa⁴ is tyrosine. That is, they comprise the amino acid sequence SEQ ID NO: 4 (HAEGTFTSDVSSY). These peptides are, in particular, for the topical treatment and/or prevention of DR.

In another embodiment the peptide according to the invention is a mammal glucagon-like peptide-1. This peptide includes at its N-terminal end (N-terminal region) the sequence identified as SEQ ID NO: 4, which is maintained in most mammals, such as humans, pigs and monkeys. In addition, this is the sequence which is mostly recognized by the GLP-1R.

Thus, in a preferred embodiment, the peptide according to the invention consists in the human glucagon-like peptide-1 of amino acid sequence SEQ ID NO: 2, corresponding to HAEGTFTSDVSSYLEGQAAKEFIAWLVKGR-NH₂, and variations of this human peptide. Also, this peptide according to the invention can be referred as the natural glucagon-like peptide-1 (7-36)amide. In some embodiments, this peptide according to the invention can be referred as the natural glucagon-like peptide-1 (7-36)amide available as acetate.

In another preferred embodiment, the peptide according to the invention consists in the human glucagon-like peptide-1 of amino acid sequence SEQ ID NO: 3, corresponding to HAEGTFTSDVSSYLEGQAAKEFIAWLVKGRG, and variations of this human peptide. Also, this peptide can be referred as the human glucagon-like peptide-1 (7-37).

The variations relate to mutations between individuals, meanwhile these mutations do not affect the interaction with the GLP-1R, and do not deprive the peptide of acting through this receptor (in particular as agonist or activator of the subsequent signalling pathway leading to neuroprotection or to the lowering of blood glucose levels). By “mutations” is to be understood any deletion of one or two amino acids, and a substitution or addition of a conservative amino acid.

Thus, the present invention also encompasses mammal glucagon-like peptide-1 (7-37) or analogues thereof for use in the topical (ocular) treatment of retinal neurodegenerative diseases, in particular of DR, wherein the analogue of glucagon-like peptide-1 (7-37) is a peptide that comprises at least one of the following modifications:
a) a deletion of at least one amino acid residue of the glucagon-like peptide-1 (7-37);
b) at least the substitution of one amino acid residue of the glucagon-like peptide-1 (7-37) by another amino acid residue; and
c) the addition of at least one amino acid residue at the C-terminal end of the glucagon-like peptide-1 (7-37), meanwhile they include at the N-terminal region the amino acid sequence SEQ ID NO: 1. Said analogues are, in addition peptide agonists of the human glucagon-like peptide-1 receptor, being able to stimulate formation of cAMP when tested in front of the receptor.

Alternatively, in another embodiment the peptide is:
(a) a peptide comprising or consisting of the amino acid sequence SEQ ID NO: 2 or a pharmaceutically acceptable salt thereof; or, alternatively,
(b) a peptide comprising or consisting of the amino acid sequence SEQ ID NO: 3 or a pharmaceutically acceptable salt thereof; or, alternatively,
(c) a peptide with an amino acid sequence having at least 85% of identity degree with SEQ ID NO: 2, 3, or a pharmaceutically acceptable salt thereof, provided that the N-terminal region is as defined in the first aspect of the invention, or, alternatively,
(d) a peptide with a sequence length up to 50 amino acids comprising an amino acid sequence having at least 85% of identity degree with SEQ ID NO: 2, 3, or a pharmaceutically acceptable salt thereof, provided that the N-terminal region is as defined in the first aspect of the invention; or, alternatively,
(e) a fragment of a peptide having at least 85% of identity degree with SEQ ID NO: 2, or a pharmaceutically acceptable salt thereof, provided that the fragment has an amino acid length from 14 to 29 amino acids and includes the N-terminal region as defined in the first aspect of the invention; or, alternatively,
(f) a fragment of a peptide having at least 85% of identity degree with SEQ ID NO: 3, or a pharmaceutically acceptable salt thereof, provided that the fragment has an amino acid length from 14 to 30 amino acids and includes the N-terminal region as defined in the first aspect of the invention.

In another embodiment of the first aspect of the invention, the peptide or salt thereof is a peptide which has an identity of 85%, 86%, 87%, 88%, 89%, 90%, 91%, 92%, 93%, 94%, 95%, 96%, 97%, 98%, 99% or 100% with respect to SEQ ID NO: 2 or SEQ ID NO: 3. In another embodiment of the first aspect of the invention, the peptide or salt thereof is a peptide which has an identity of 100% with respect to sequence SEQ ID NO: 2. In another embodiment of the first aspect of the invention, the peptide or salt thereof is a peptide which has an identity of 100% with respect to sequence SEQ ID NO: 3. In another embodiment of the first aspect of the invention, the peptide is a pharmaceutically acceptable salt of the sequence SEQ ID NO: 2, particularly is an acetate salt of the sequence SEQ ID NO: 2. Alternatively, in another embodiment of the first aspect of the invention the peptide is the sequence SEQ ID NO: 2.

In the present invention the term “identity” refers to the percentage of residues that are identical in the two sequences when the sequences are optimally aligned. If, in the optimal alignment, a position in a first sequence is occupied by the same amino acid residue as the corresponding position in the second sequence, the sequences exhibit identity with respect to that position. The level of identity between two sequences (or “percent sequence identity”) is measured as a ratio of the number of identical positions shared by the sequences with respect to the size of the sequences (i.e., percent sequence identity=(number of identical positions/total number of positions)×100). In the context of the present invention, the peptide having an amino acid sequence identity degree of at least 85% with respect SEQ ID NO: 2 or 3 will retain the N-terminal region as defined in the first aspect of the invention and any of the embodiments above.

A number of mathematical algorithms for rapidly obtaining the optimal alignment and calculating identity between two or more sequences are known and incorporated into a number of available software programs. Examples of such programs include the MATCH-BOX, MULTAIN, GCG, FASTA, and ROBUST programs for amino acid sequence analysis, among others. Preferred software analysis programs include the ALIGN, CLUSTAL W, and BLAST programs (e.g., BLAST 2.1, BL2SEQ, and later versions thereof).

For amino acid sequence analysis, a weight matrix, such as the BLOSUM matrixes (e.g., the BLOSUM45, BLOSUM50, BLOSUM62, and BLOSUM80 matrixes), Gonnet matrixes, or PAM matrixes (e.g., the PAM30, PAM70, PAM120, PAM160, PAM250, and PAM350 matrixes), are used in determining identity.

The BLAST programs provide analysis of at least two amino acid sequences, either by aligning a selected sequence against multiple sequences in a database (e.g., GenSeq), or, with BL2SEQ, between two selected sequences. BLAST programs are preferably modified by low complexity filtering programs such as the DUST or SEG programs, which are preferably integrated into the BLAST program operations. If gap existence costs (or gap scores) are used, the gap existence cost preferably is set between about −5 and −15. Similar gap parameters can be used with other programs as appropriate. The BLAST programs and principles underlying them are further described in, e.g., Altschul et al., “Basic local alignment search tool”, 1990, J. Mol. Biol, v. 215, pages 403-410.

For multiple sequence analysis, the CLUSTAL W program can be used. The CLUSTAL W program desirably is run using “dynamic” (versus “fast”) settings. Amino acid sequences are evaluated using a variable set of BLOSUM matrixes depending on the level of identity between the sequences. The CLUSTAL W program and underlying principles of operation are further described in, e.g., Higgins et al., “CLUSTAL V: improved software for multiple sequence alignment”, 1992, CABIOS, 8(2), pages 189-191.

In particular mammal glucagon-like peptide-1 (7-37) or analogues thereof are usable in the treatment and/or prevention of retinal neurodegenerative diseases, in particular of DR. The peptides, when applied topically in the eye, act as neuroprotector agents (avoiding neurodegeneration in case of preventive treatment).

In another embodiment of the present invention, the ophthalmic pharmaceutical composition of the first aspect has a pH value between 4.1 and 4.8 and preferably the pH value is between 4.2 and 4.7.

The inventors have found that the pH can affect the chemical stability, potency, and effectiveness of the peptides of the invention. An optimum pH avoids adverse effects, ensures that drugs will produce an optimum therapeutic effect, and ensures all components' roles are optimized. Buffers are used in ophthalmic compositions when the pH is critical and must be within a certain range. Herein, the term “buffering agent” refers to a mixture of an acid (usually a weak acid, e.g. acetic acid, citric acid) and its conjugate base (e.g. an acetate or citrate salt, for example, sodium acetate, sodium citrate) in a ratio that will resist pH change if dissolved in an aqueous solution. Ideally, the pH of ophthalmic drops should be equivalent to that of tear fluid, which is 7.4. However, the decision to add a buffering agent should be based on stability considerations. The pH selected should be the optimum for both stability of the active pharmaceutical ingredient and physiological tolerance.

In another embodiment of the present invention, the osmolality of the ophthalmic pharmaceutical composition of the first aspect ranges from 1 to 150 mOsm/kg. In a preferred embodiment the osmolality ranges from 1 to 90 mOsm/kg, preferably from 1 to 80 mOsm/kg, more preferably, from 1 to 70 mOsm/kg, even more preferably from 1 to 50 mOsm/kg.

In a preferred embodiment the osmolality ranges from 1 to 10 mOsm/kg. In another preferred embodiment, the osmolality ranges from 85 to 150 mOsm/kg.

Tonicity refers to the osmotic pressure exerted by salts in aqueous solution. An ophthalmic solution is isotonic with another solution when the magnitudes of the colligative properties of the solutions are equal. An ophthalmic solution is considered isotonic when its tonicity is equal to that of 0.9% sodium chloride solution (290 mOsm/kg). Since human tears are isotonic and very similar to 0.9% sodium chloride solution, it was thought that tonicity was important for ophthalmic preparations.

In the present invention, inventors surprisingly found that the ophthalmic compositions of the present invention are well tolerated, stable and effective even not being isotonic and having a pH lower than 7.4, which is contrary to the majority of ophthalmic compositions in the prior-art.

Thus, the osmolality of the solution ranges between 0.5 and 200 mOsm/kg. The osmolality of a real solution corresponds to the molality of an ideal solution containing non-dissociating solutes and is expressed in osmoles or milliosmoles per kilogram of solvent (Osmol per kg or mOsmol per kg, respectively), a unit that is similar to the molality of the solution. Thus, osmolality is a measure of the osmotic pressure exerted by a real solution across a semipermeable membrane. The osmolality of a solution is commonly determined by the measurement of the freezing point depression of the solution.

The apparatus, an osmometer for freezing point depression measurement, consists of the following: a means of cooling the container used for the measurement; a resistor sensitive to temperature (thermistor), with an appropriate current- or potential-difference measurement device that may be graduated in temperature change or in osmolality; and a means of mixing the sample. The osmolality was measured using the method according to Pharmacopeial Forum: Volume No. 34(1) Page 157, chapter <785> Osmolality and Osmolarity.

In another embodiment of the present invention, the ophthalmic pharmaceutical composition of the first aspect further comprises at least one compound selected from the group consisting of a stabilizing agent, a viscosifying agent, a buffering agent and mixtures thereof.

The stabilizing agent is introduced in small amount in order to maintain both pH and osmolality low in particular lower to what is generally done by the state of the art. In particular, in another embodiment of the present invention, the stabilizing agent is aspartic acid or glutamic acid. In a preferred embodiment, wherein the ratio in weight of stabilizing agent in the composition versus the peptide is in the range of from 1:5 to 1:50, preferably from 1:8 to 1:30, more preferably 1:10 to 1:20.

Herein, a “stabilizing agent” refers to a component which facilitates maintenance of the structural integrity of the biopharmaceutical drug, particularly during storage (especially when exposed to stress) and in solution. This stabilising effect may arise for a variety of reasons, though typically such stabilisers may act as osmolytes which mitigate against protein denaturation or aggregation. Typical stabilisers include amino acids (i.e. free amino acids not part of a peptide or protein—e.g. glycine, arginine, histidine, aspartic acid, lysine) and sugar stabilisers, such as a sugar polyol (e.g. mannitol, sorbitol), and/or a disaccharide (e.g. trehalose, sucrose, maltose, lactose).

In another embodiment of the present invention, the buffering agent is acetic acid/acetate or citric acid/citrate. In a preferred embodiment, the total amount of buffering agent in the composition is from 0.05% to 5.0% w/w, more preferably from 0.08% to 2.0% w/w, more preferably 0.1% to 1.5%. In another preferred embodiment, the strength of the buffering agent is in the range between 20 mM and 100 mM, more preferably between 30 mM to 70 mM.

Herein; the “strength” refers to ionic strength which is a measure of the concentration of ions in that solution. It is based on the dissociation that suffers salts, acid and bases when are in an aqueous solution. It is expressed in concentration units, such as molar concentration.

In another embodiment of the present invention, the viscosifying agent is selected from hydroxyethyl cellulose, methylcellulose, hydroxypropyl methylcellulose, carboxymethyl cellulose, hydroxypropyl cellulose, polyvinyl alcohol, polyvinylpyrrolidone, sodium hyaluronate, carbopol, polyacrylamide, sodium chondroitin sulfate and mixtures thereof. In a preferred embodiment, wherein the viscosifying agent is hydroxypropyl methylcellulose or polyvinylpyrrolidone.

Herein, a “viscosifying agent” refers to a component which thicken ophthalmic liquid vehicles, especially aqueous solutions, to increase contact time of the drug with the eye and minimize drainage into the nasolacrimal system. Viscosity increases drug absorption and therapeutic effects. The present composition can also include a viscosifying agent.

In another preferred embodiment of the present invention, the total amount of viscosifying agent in the composition is from 0.1% to 5% w/w, preferably from 0.3% to 4% w/w, more preferably from 0.5% to 3% w/w.

In another embodiment of the present invention, the viscosity is in the range between 1 and 50 cSt at room temperature and pressure, preferably between 1 and 20 cSt, more preferably between 1 and 10 cSt. The viscosity was measured using the capillary viscometer method described European Pharmacopeia 7.0 2.2.9.

In another embodiment of the present invention, the ophthalmic pharmaceutical composition of the first aspect further comprises an effective amount of a preservative. Commonly known preservatives are contemplated herein, including a detergent preservative, an oxidizing preservative, and an ionic-buffered preservative. In a preferred embodiment, the preservative is selected from edetate sodium, benzalkonium chloride, centrimonium chloride, sodium perborate, stabilized oxychloro complex, sorbic acid, thimerosal, polyquarternium-1, polyhexamethylene biguanide, chlorobutanol, phenylethyl alcohol, methylparaben, propylparaben, a combination of boric acid, sorbic acid, and propylene glycol; and mixtures thereof, more preferably the preservative is benzalkonium chloride.

In another preferred embodiment, the total amount of preservative in the composition is from 0.005% to 0.5% w/w, preferably from 0.008% to 0.3% w/w, more preferably from 0.01% to 0.1% w/w.

Herein, a “preservative” refers to substances that prevent or inhibit microbial growth and extend the shelf life of the drug products, such as in ophthalmic solutions. The use of preservatives in topical ophthalmic treatments is ubiquitous for any product that is to be used more than once by the patient as they prevent any microbes that may enter into the product after its first use from allowing those microbes to grow and infect the patient on a later use of the product.

Antimicrobial preservatives are not found in single use vials of ophthalmic solutions since they are manufactured aseptically or are sterilised, and the products are used once and the dispenser is thrown away.

For ophthalmic preparations that must be sterilized, an appropriate and validated method of sterilization should be determined on the basis of the characteristics of the particular product and container. Filtration of the preparation through a 0.22 pm filter into a sterile final container is a commonly used method.

In another embodiment of the present invention, the concentration of the peptide in said composition ranges between 1 and 50 mg/mL, preferably ranges between 1 and 25 mg/mL, more preferably ranges between 1 and 10 mg/mL, even more preferably ranges between 1 and 5 mg/mL.

In another embodiment of the present invention, the ophthalmic pharmaceutical composition of the first aspect is in the form of solution, such as eye drops. The administration of the peptides in the form of eye drops implies the great advantage of being easy to be used by the subject in need thereof, and non-discomfortable.

In another embodiment of the present invention, the ophthalmic pharmaceutical composition is selected from creams, lotions, unguents, emulsions, aerosols and non-aerosol sprays, gels, ointments, and suspensions.

Additionally, the compositions of the present invention may contain other ingredients, such as fragrances, colorants, and other components known in the state of the art for use in topical formulations.

Topical compositions of the present invention can be prepared according to methods well known in the state of the art. The appropriate excipients and/or carriers, and their amounts, can readily be determined by those skilled in the art according to the type of formulation being prepared.

In a second aspect, the present invention relates to a lyophilizate.

In one embodiment of the second aspect, the lyophilizate is in the form of lyophilized cake or powder.

In another embodiment of the second aspect, the water content of the lyophilizate is below 5.0% by weight of the total amount of the lyophilizate, preferably is below 3.0% by weight of the total amount of the lyophilizate, more preferably is below 2.0% by weight of the total amount of the lyophilizate.

In a third aspect, the present invention relates to a process for preparing the ophthalmic pharmaceutical composition of the first aspect.

In one embodiment of the third aspect, said process comprises in step a) freeze drying a solution comprising the steps of freezing the solution, primary drying and secondary drying, and wherein the freeze drying is less than 40 hours long, preferably between 10 and 35 hours long, and more preferably between 15 and 30 hours long from the initial step of freezing the solution until the end of the secondary drying.

In another embodiment of the third aspect, said process in step a) provides the lyophilizate of the second aspect.

In a fourth aspect, the present invention relates to an ophthalmic pharmaceutical composition obtainable by the process of the third aspect.

In a fifth aspect, the present invention relates to a kit comprising the lyophilizate and a physiologically acceptable vehicle composition comprising one or more pharmaceutically acceptable excipients or carriers, for reconstituting the peptide.

The kit optionally further includes instructions for performing the reconstitution of the lyophilizate to obtain the composition of the invention.

The lyophilizate and the vehicle can be contained in separated containers (vials) or, alternatively, in a two-compartment container (vial), wherein one compartment contains the lyophilizate and the other compartment contains the vehicle.

In one embodiment of the fifth aspect, the physiologically acceptable vehicle comprises at least one viscosifying agent and optionally at least one preservative.

In a sixth aspect, the present invention relates to a kit comprising the ophthalmic pharmaceutical composition of the first and fourth aspect, a container for holding the pharmaceutical composition and a drop dispenser adapted for administering about 10 to 100 μl volume of the composition per drop, preferably about 10 to 50 μl volume, more preferably about 20 to 40 μl volume.

In another embodiment of the fifth and sixth aspects, the container and/or drop dispenser is manufactured from a thermoplastic material or glass, preferably the thermoplastic material is selected from polyethylene or polypropylene. In a preferred embodiment of the fifth aspect, the container is manufactured from polypropylene and the drop dispenser is manufactured from a polyethylene selected from low or high density polyethylene. In another preferred embodiment of the fifth aspect, the container and the drop dispenser are manufactured from glass.

The final container should be appropriate for the ophthalmic product and its intended use and should not interfere with the stability and efficacy of the preparation.

The protection of retinal neurodegeneration detected by means of several ophthalmological examinations represents a good approach for treating DR. In the early stages of DR neurodegeneration exists (which can be detected by the loss of both chromatic discrimination and contrast sensitivity, glial activation and apoptosis of neural cells). The ophthalmic pharmaceutical composition of the invention is useful in retinal degenerative diseases, in particular DR, especially in early stages when no treatment is indicated and only the follow-up is recommended until more advances stages of DR are established (clinically significant diabetic macular edema and/or proliferative diabetic retinopathy).

Treatment in the early stages of DR has the real advantage that further complications are avoided, namely microaneurysms, microhemorrhages, hard exudates, neovascularization, capillary occlusion, and breakdown of the blood retinal barrier (BRB).

In another embodiment of the present invention, the ophthalmic pharmaceutical composition of the first or fourth aspect or the kit of the fifth aspect for use in the topical eye treatment and/or prevention of a retinal neurodegenerative disease.

In a preferred embodiment, the retinal neurodegenerative disease is selected from the group consisting of diabetic retinopathy (DR), age-related macular degeneration, glaucoma and retinitis pigmentosa. In a more preferred embodiment, the retinal neurodegenerative disease is diabetic retinopathy.

In another more preferred embodiment, the ophthalmic pharmaceutical composition of the first or fourth aspect or the kit of the fifth aspect for use in the topical treatment of early stages of the diabetic retinopathy.

In another more preferred embodiment, the composition being administered from one to four times per day, preferably once-daily, preferably twice-daily, preferably three times per day, preferably four times per day.

The present invention will now be described in more detail with reference to the following Examples, which should in no way be construed to be limiting the scope of the present invention.

Further aspects and embodiments of the present invention are described in the following clauses below:

Clause 1. An ophthalmic pharmaceutical composition comprising:

a peptide with a sequence length from 13 to 50 amino acids, the N-terminal region of said peptide consisting in the sequence:
HXaa¹EGTFTSDXaa²SXaa³Xaa⁴(SEQ ID NO: 1) wherein:
   Xaa¹ is an amino acid selected from alanine and glycine;
   Xaa² is an amino acid selected from valine and leucine;
   Xaa³ is an amino acid selected from serine and lysine;
   Xaa⁴ is an amino acid selected from tyrosine and glutamine; and
histidine is the N-terminal residue, and

one or more pharmaceutically acceptable excipients or carriers;

wherein the pH value of the solution is between 4.0 and 4.8 and the osmolality ranges between 0.5 and 200 mOsm/kg.

Clause 2. The ophthalmic pharmaceutical composition according to the preceding clause, wherein the sequence length is from 30 to 40 amino acids.

Clause 3. The ophthalmic pharmaceutical composition according to any one of the preceding clauses, wherein Xaa¹ is alanine, Xaa² is valine, Xaa³ is serine, and Xaa⁴ is tyrosine.

Clause 4. The ophthalmic pharmaceutical composition according to any one of the preceding clauses, wherein the peptide is a mammal glucagon-like peptide-1, or a pharmaceutically acceptable salt thereof.

Clause 5. The ophthalmic pharmaceutical composition according to any one of the preceding clauses, wherein the peptide is:
(a) a peptide comprising or consisting of the amino acid sequence SEQ ID NO: 2 or a pharmaceutically acceptable salt thereof; or, alternatively,
(b) a peptide comprising or consisting of the amino acid sequence SEQ ID NO: 3 or a pharmaceutically acceptable salt thereof; or, alternatively,
(c) a peptide with an amino acid sequence having at least 85% of identity degree with SEQ ID NO: 2, 3, or a pharmaceutically acceptable salt thereof, provided that the N-terminal region is as defined in the first aspect of the invention, or, alternatively,
(d) a peptide with a sequence length up to 50 amino acids comprising an amino acid sequence having at least 85% of identity degree with SEQ ID NO: 2, 3, or a pharmaceutically acceptable salt thereof, provided that the N-terminal region is as defined in the first aspect of the invention; or, alternatively,
(e) a fragment of a peptide having at least 85% of identity degree with SEQ ID NO: 2, or a pharmaceutically acceptable salt thereof, provided that the fragment has an amino acid length from 14 to 29 amino acids and includes the N-terminal region as defined in the first aspect of the invention; or, alternatively,
(f) a fragment of a peptide having at least 85% of identity degree with SEQ ID NO: 3, or a pharmaceutically acceptable salt thereof, provided that the fragment has an amino acid length from 14 to 30 amino acids and includes the N-terminal region as defined in the first aspect of the invention.

Clause 6. The ophthalmic pharmaceutical composition according to any one of the clauses 1 to 4, wherein the peptide is a pharmaceutical acceptable salt of the sequence SEQ ID NO: 2, particularly an acetate salt of the sequence SEQ ID NO: 2; or, alternatively, the peptide is the sequence SEQ ID NO: 2.

Clause 7. The ophthalmic pharmaceutical composition according to any one of the preceding clauses, wherein the pH value is between 4.1 and 4.8, preferably the pH value is between 4.2 and 4.7.

Clause 8. The ophthalmic pharmaceutical composition according to any one of the preceding clauses, wherein the osmolality ranges from 1 to 150 mOsm/kg.

Clause 9. The ophthalmic pharmaceutical composition according to any one of the preceding clauses, wherein the osmolality ranges from 1 to 90 mOsm/kg.

Clause 10. The ophthalmic pharmaceutical composition according to any one of the preceding clauses, wherein the osmolality ranges from 1 to 80 mOsm/kg.

Clause 11. The ophthalmic pharmaceutical composition according to any one of the preceding clauses, wherein the osmolality ranges from 1 to 70 mOsm/kg.

Clause 12. The ophthalmic pharmaceutical composition according to any one of the preceding clauses, wherein the osmolality ranges from 1 to 50 mOsm/kg.

Clause 13. The ophthalmic pharmaceutical composition according to any one of the preceding clauses, wherein the osmolality ranges from 1 to 10 mOsm/kg.

Clause 14. The ophthalmic pharmaceutical composition according to any of the clauses 1 to 9, wherein the osmolality ranges from 85 to 150 mOsm/kg.

Clause 15. The ophthalmic pharmaceutical composition according to any one of the preceding clauses, wherein at least one of the one or more pharmaceutically acceptable excipients or carriers is selected from the group consisting of a stabilizing agent, a viscosifying agent, a buffering agent and mixtures thereof.

Clause 16. The ophthalmic pharmaceutical composition according to the preceding clause, wherein the stabilizing agent is aspartic acid or glutamic acid.

Clause 17. The ophthalmic pharmaceutical composition according to any one of the two preceding clauses, wherein the ratio in weight of stabilizing agent in the composition versus the peptide is in the range of from 1:5 to 1:50, preferably from 1:8 to 1:30, more preferably 1:10 to 1:20.

Clause 18. The ophthalmic pharmaceutical composition according to any one of the three preceding clauses, wherein the buffering agent is acetic acid/acetate or citric acid/citrate.

Clause 19. The ophthalmic pharmaceutical composition according to any one of the four preceding clauses, wherein the total amount of buffering agent in the composition is from 0.05% to 5.0% w/w, preferably from 0.08% to 2.0% w/w, more preferably 0.1% to 1.5%.

Clause 20. The ophthalmic pharmaceutical composition according to any one of the five preceding clauses, wherein the strength of the buffering agent is in the range between 20 mM and 100 mM, preferably between 30 mM to 70 mM.

Clause 21. The ophthalmic pharmaceutical composition according to any one of the clauses six preceding clauses, wherein the viscosifying agent is selected from hydroxyethyl cellulose, methylcellulose, hydroxypropyl methylcellulose, carboxymethyl cellulose, hydroxypropyl cellulose, polyvinyl alcohol, polyvinylpyrrolidone, sodium hyaluronate, carbopol, polyacrylamide, sodium chondroitin sulfate and mixtures thereof.

Clause 22. The ophthalmic pharmaceutical composition according to the preceding clause, wherein the viscosifying agent is hydroxypropyl methylcellulose or polyvinylpyrrolidone.

Clause 23. The ophthalmic pharmaceutical composition according to any one of the two preceding clauses or clause 11, wherein the total amount of viscosifying agent in the composition is from 0.1% to 5% w/w, preferably from 0.3% to 4% w/w, more preferably from 0.5% to 3% w/w.

Clause 24. The ophthalmic pharmaceutical composition according to any one of the preceding clauses, wherein the viscosity is in the range between 1 and 50 cSt at room temperature and pressure, preferably between 1 and 20 cSt, more preferably between 1 and 10 cSt.

Clause 25. The ophthalmic pharmaceutical composition according to the any one of the preceding clauses further comprising an effective amount of a preservative.

Clause 26. The ophthalmic pharmaceutical composition according to the preceding clause, wherein the preservative is selected from edetate sodium, benzalkonium chloride, centrimonium chloride, sodium perborate, stabilized oxychloro complex, sorbic acid, thimerosal, polyquarternium-1, polyhexamethylene biguanide, chlorobutanol, phenylethyl alcohol, methylparaben, propylparaben, a combination of boric acid, sorbic acid, and propylene glycol; and mixtures thereof.

Clause 27. The ophthalmic pharmaceutical composition according any one of the two preceding clauses, wherein the preservative is benzalkonium chloride.

Clause 28. The ophthalmic pharmaceutical composition according to any one of the three preceding clauses, wherein the total amount of preservative in the composition is from 0.005% to 0.5% w/w, preferably from 0.008% to 0.3% w/w, more preferably from 0.01% to 0.1% w/w.

Clause 29. The ophthalmic pharmaceutical composition according to any one of the preceding clauses, wherein the concentration of the peptide in said composition ranges between 1 and 50 mg/mL, preferably ranges between 1 and 25 mg/mL, more preferably ranges between 1 and 10 mg/mL, even more preferably ranges between 1 and 5 mg/mL.

Clause 30. The ophthalmic pharmaceutical composition according to any one of the preceding clauses characterized in that the composition is in the form of solution.

Clause 31. A lyophilizate obtainable by lyophilization of a solution comprising:
a) a pharmaceutically effective amount of the peptide as defined in any one of the clauses 1 to 6 and/or a pharmaceutically acceptable salt thereof,
b) a pharmaceutically acceptable amount of stabilizing agent or buffering agent, and
c) water

wherein said lyophilizate is suitable for preparing the ophthalmic pharmaceutical composition according to any one of the preceding clauses by reconstitution.

Clause 32. A lyophilizate comprising the peptide as defined in any one of the clauses 1 to 6 or a pharmaceutically acceptable salt thereof, and a pharmaceutically acceptable amount of a stabilizing agent or buffering agent, wherein said lyophilizate is suitable for preparing the ophthalmic pharmaceutical composition according to any one of the preceding clauses by reconstitution.

Clause 33. The lyophilizate according to the two preceding clauses, wherein the lyophilizate is in the form of lyophilized cake or powder.

Clause 34. The lyophilizate according to any one of the three preceding clauses, wherein the water content of the lyophilizate is below 5.0% by weight of the total amount of the lyophilizate, preferably is below 3.0% by weight of the total amount of the lyophilizate, more preferably is below 2.0% by weight of the total amount of the lyophilizate.

Clause 35. A process for preparing the ophthalmic pharmaceutical composition according to according to any one of the clauses 1 to 30, which comprises the step of reconstituting the lyophilizate as defined in any one of the clauses 31-34, with an aqueous vehicle composition comprising one or more pharmaceutically acceptable carriers or excipients, particularly an aqueous vehicle composition comprising at least one viscosifying agent and optionally at least one preservative.

Clause 36. A process for preparing the ophthalmic pharmaceutical composition according to any one of the clauses 1 to 30, which comprises:
a) providing a lyophilizate comprising a pharmaceutically effective amount of the peptide as defined in any one of the clauses 1 to 6 and/or a pharmaceutically acceptable salt thereof and a pharmaceutically acceptable amount of stabilizing agent or buffering agent,
b) providing a vehicle composition comprising one or more pharmaceutically acceptable excipients or carriers, such as at least one viscosifying agent and optionally at least one preservative; and
c) reconstituting the lyophilizate of step a) with the vehicle composition of step b) to form an ophthalmic pharmaceutical composition.

Clause 37. The process according to the preceding clause, wherein said process comprises in step a) freeze drying a solution comprising the steps of freezing the solution, primary drying and secondary drying, and wherein the freeze drying is less than 40 hours long, preferably between 10 and 35 hours long, and more preferably between 15 and 30 hours long from the initial step of freezing the solution until the end of the secondary drying.

Clause 38. An ophthalmic pharmaceutical composition obtainable by the process as defined in any one of clauses 35 to 37.

Clause 39. A kit comprising the lyophilizate as defined in any one of clauses 31 to 34 and a physiologically acceptable vehicle composition comprising one or more pharmaceutically acceptable excipients or carriers, for reconstituting the peptide.

Clause 40. The kit according to the preceding clause, wherein the physiologically acceptable vehicle composition comprises at least one viscosifying agent and optionally at least one preservative.

Clause 41. A kit comprising the ophthalmic pharmaceutical composition as defined in any one of clauses 1 to 30 or 38, a container for holding the pharmaceutical composition and a drop dispenser adapted for administering a volume, for example, about 10 to 100 μl volume of the composition per drop, preferably about 10 to 50 μl volume, more preferably about 20 to 40 μl volume.

Clause 42. The kit according to the preceding clause, wherein the container and/or drop dispenser is manufactured from a thermoplastic material or glass, preferably the thermoplastic material is selected from polyethylene or polypropylene.

Clause 43. The kit according to any one of the two preceding clauses, wherein the container is manufactured from polypropylene and the drop dispenser is manufactured from a polyethylene selected from low or high density polyethylene.

Clause 44. An ophthalmic pharmaceutical composition according to any one of clauses 1 to 30 or 38 or the kit according to any one of clauses 39 to 42 for use in the topical eye treatment and/or prevention of a retinal neurodegenerative disease.

Clause 45. The ophthalmic pharmaceutical composition or the kit for use according to the preceding clause, wherein the retinal neurodegenerative disease is selected from the group consisting of diabetic retinopathy (DR), age-related macular degeneration, glaucoma and retinitis pigmentosa.

Clause 46. The ophthalmic pharmaceutical composition or the kit for use according to any one of the two preceding clauses, wherein the retinal neurodegenerative disease is diabetic retinopathy.

Clause 47. The ophthalmic pharmaceutical composition or the kit for use according to any one the three preceding clauses for use in the topical treatment of non-proliferative diabetic retinopathy.

Clause 48. The ophthalmic pharmaceutical composition or the kit for use according to any one of the four preceding clauses, wherein the composition being administered from one to four times per day, preferably once-daily, preferably twice-daily, preferably three times per day, preferably four times per day.

### EXAMPLES

### Example 1. Preparation of an Ophthalmic Ready to use Solution of Acetic/Acetate Buffer at pH 4.4 with GLP-1 (7-36) Amide at a Concentration of 2 mg/mL

An ophthalmic aqueous composition at 2 mg/ml of synthetic human glucagon—like peptide (7-36) amide was manufactured at a 60 mL scale.

| GLP-1 (7-36) amide base concentration | 2 mg/mL |
|---|---|
| Component | % (w/w) |
| GLP-1 (7-36) amide acetate salt | 0.22* |
| [GLP-1 (7-36) amide base* | [0.20] |
| Sodium acetate | 0.13 |
| Ammonium acetate | 0.08 |
| Acetic acid | 0.25 |
| Polyvinylpyrrolidone | 1.00 |
| Water for irrigation | 98.32 |

*Taking into account a Batch 020217 peptide content in the acetate salt of 90.78%. Supplier of the peptide: Hemmo Pharmaceuticals Pvt. Ltd

136 mg of sodium acetate and 77 mg of ammonium acetate were weighted and adjusted to a volume of 100 mL with water for irrigation in a ready to use 250 mL sterile container. The mixture was stirred until complete dissolution. 0.25 mL of acetic acid is then added to the solution. The pH of the acetic/acetate solution was 4.4.

99 g of the previous solution were sampled in a new reactor equipped with magnetic stirring. 1 g of polyvinylpyrrolidone K90, as viscosifying agent, was weighed and added stepwise into the reactor to the 99 g of the solution previously prepared for 30 minutes to ensure good dissolution.

59.87 g of the previous solution were weighted and added in a reactor equipped with magnetic stirring, 133.5 mg of GLP-1 (7-36) amide as acetate salt (batch: 020217, pure peptide content p90.78%) were weighted and added under stirring until complete dissolution. The pH of the final solution was 4.5.

The final solution was filtered through polyvinylidene difluoride (PVDF) filter membrane with 0.22 μm pore size. Type I glass vials (2 mL) were filled with 2 mL of the previously filtered solution. The vials were closed using 13 mm bromobutyl rubber and sealed with 13 mm aluminium cap. 28 vials were obtained reaching a final yield around 93% w/w.

The ophthalmic solution obtained was then characterized. The solution had a clear solution aspect. Moreover, pH (Metrohm 780), osmolality (Osmomat 030-D) and GLP-1 (7-36) amide content and purity by RP-HPLC were determined over time up to 12 months at two storage conditions 5° C. and 25° C./60% RH. The RP-HPLC method used in all the examples is described hereafter.

| Column: | YMC Pack-Pro C18 250 × 4.6 mm, 5 μm, 12 nm | | | | |
|---|---|---|---|---|---|
| Organic mobile phase | 0.07% TFA in Water/ACN (65:35) | | | | |
| A: | | | | | |
| Aqueous mobile phase | 0.1% TFA in purified Water | | | | |
| Elution mode: | Gradient | | | | |
| Gradient: | (min) | 0 | 32 | 33 | 40 |
| | % MPA: | 30.8 | 76.3 | 30.8 | 30.8 |
| | % MPB: | 69.2 | 23.7 | 69.2 | 69.2 |

| | |
|---|---|
| Flow: | 1.0 mL/min |
| Injection volume: | 40 μL |
| Detection: | 220 nm |
| Temp. autosampler: | 10° C. |
| Temp. Column: | 45° C. |

Table below shows the visual aspect, pH, osmolality, GLP-1 (7-36) amide content and purity over time up to 12 months at two storage conditions 5° C. and 25° C./60% RH.

| | | t = 1 M | | t = 12 M | |
|---|---|---|---|---|---|
| | | | 25° C./ | | 25° C./ |
| Parameters | t0 | 5° C. | 60% RH | 5° C. | 60% RH |
| Visual aspect | Solution | Solution | Solution | Solution | Solution |
| pH | 4.5 | 4.5 | 4.5 | 4.5 | 4.6 |
| Osmolality | 99.3 | 100.3 | 97.0 | 98.5 | 97.0 |
| (mOsm/Kg) | | | | | |
| GLP-1 (7-36) | 0.20 | 0.19 | 0.15 | 0.15 | 0.06 |
| amide | | | | | |
| content (%) | | | | | |
| Purity (%) | 98.8 | 96.0 | 84.3 | 84.9 | 49.2 |

### Example 2. Preparation of an Ophthalmic Ready to use Solution of Aspartic Acid with GLP-1 (7-36) Amide at a Concentration of 2 mg/mL

An ophthalmic aqueous composition at 2 mg/ml of synthetic human glucagon—like peptide (7-36) amide was manufactured at a 60 mL scale.

| GLP-1 (7-36) amide base concentration | 2 mg/mL |
|---|---|
| Component | % (w/w) |
| GLP-1 (7-36) amide acetate salt | 0.22* |
| [GLP-1 (7-36) amide base*] | [0.20] |
| Aspartic acid | 0.016 |
| Polyvinylpyrrolidone | 1.00 |
| Water for irrigation | 98.76 |

*Taking into account a Batch 020217 peptide content in the acetate salt of 90.78%. Supplier of the peptide: Hemmo Pharmaceuticals Pvt. Ltd

16.1 mg of aspartic acid were weighted and then adjusted to a volume of 100 mL with water for irrigation in a ready to use 250 mL sterile container. The mixture was stirred until complete dissolution.

99 g of the previous solution were sampled in a new reactor equipped with magnetic stirring. 1 g of polyvinylpyrrolidone K90, as viscosifying agent, was weighed and added stepwise into the reactor to the 99 g of the solution previously prepared for 30 minutes to ensure good dissolution.

59.87 g of the previous solution were weighted and added in a reactor equipped with magnetic stirring, 133.5 mg of GLP-1 (7-36) amide as acetate salt (batch: 020217, pure peptide content #90.78%) were weighted and added in the previous weighted solution and stirred to get a complete dissolution. The pH of the final solution was 4.5.

The final solution was filtered through polyvinylidene difluoride (PVDF) filter membrane with 0.22 μm pore size. Type I glass vials (2 mL) were filled with 2 mL of the previously filtered solution. The vials were closed using 13 mm bromobutyl rubber and sealed with 13 mm aluminium cap. 29 vials were obtained reaching a final yield around 97% w/w.

The ophthalmic solution obtained was then characterized. The solution had a clear solution aspect. Moreover, pH, osmolality and GLP-1 (7-36) amide content and purity by RP-HPLC were determined over time up to 12 months at two storage conditions 5° C. and 25° C./60% RH. The RP-HPLC method used is described in Example-1.

Table below shows the visual aspect, pH, osmolality, GLP-1 (7-36) amide content and purity over time up to 12 months at two storage conditions 5° C. and 25° C./60% RH.

| | | t = 1 M | | t = 6 M | | t = 12 M | |
|---|---|---|---|---|---|---|---|
| | | | 25° C./ | | 25° C./ | | 25° C./ |
| Parameters | t0 | 5° C. | 60% RH | 5° C. | 60% RH | 5° C. | 60% RH |
| Visual aspect | Solution | Solution | Solution | Solution | Solution | Solution | Solution |
| pH | 4.5 | 4.5 | 4.5 | 4.5 | 4.5 | 4.5 | 4.6 |
| Osmolality | 1.3 | 5.7 | 5.3 | 4.0 | 3.0 | 3.5 | 4.5 |
| (mOsm/Kg) | | | | | | | |
| GLP-1 (7-36) | 0.20 | 0.20 | 0.18 | 0.19 | 0.13 | 0.18 | 0.10 |
| amide content (%) | | | | | | | |
| Purity (%) | 98.9 | 98.3 | 93.7 | 95.2 | 76.5 | 93.4 | 65.2 |

### Example 3. Preparation of a Ready to use Aqueous Solution of Aspartic Acid with GLP-1 (7-36) Amide at a Concentration of 2 mg/mL and Benzalkonium Chloride as Preservative

An ophthalmic aqueous composition at 2 mg/ml of synthetic human glucagon—like peptide (7-36) amide was manufactured at a 50 mL scale.

| GLP-1 (7-36) amide base concentration | 2 mg/mL |
|---|---|
| Component | % (w/w) |
| GLP-1 (7-36) amide acetate salt | 0.22* |
| [GLP-1 (7-36) amide base*] | [0.20] |
| Aspartic acid | 0.016 |
| Polyvinylpyrrolidone | 1.00 |
| Benzalkonium chloride | 0.02 |
| Water for irrigation | 98.74 |

*Taking into account a Batch 020217 peptide content in the acetate salt of 90.78%. Supplier of the peptide: Hemmo Pharmaceuticals Pvt. Ltd

16.1 mg of aspartic acid were weighted and adjusted to a volume of 100 mL with water for irrigation in a ready to use 250 mL sterile container. The mixture was stirred until complete dissolution.

99 g of the previous solution were sampled in a new reactor equipped with magnetic stirring. 1 g of polyvinylpyrrolidone K90, as viscosifying agent, was weighed and added stepwise into the reactor to the 99 g of the solution previously prepared for 30 minutes to ensure good dissolution. Afterwards, 20.0 mg of benzalkonium chloride were added and stirred until complete dissolution.

49.90 g of the previous solution were weighted in a reactor equipped with magnetic stirring, 111.3 mg of GLP-1 (7-36) amide as acetate (batch: 020217, pure peptide content p90.78%) were weighted and added in the previous weighted solution and stirred to get a complete dissolution. The pH of the final solution was 4.4.

The final solution was filtered through polyvinylidene difluoride (PVDF) filter membrane with 0.22 μm pore size. Type I glass vials (2 mL) were filled with 2 mL of the previously filtered solution. The vials were closed using 13 mm bromobutyl rubber and sealed with 13 mm aluminium cap. 24 vials were obtained reaching a final yield around 96% w/w.

The ophthalmic solution obtained was then characterized. The solution had a clear solution aspect. Moreover, pH, osmolality and GLP-1 (7-36) amide content and purity by RP-HPLC were determined over time up to 6 weeks at two storage conditions 5° C. and 25° C./60% RH. The RP-HPLC method used is described in Example-1.

Table below shows the visual aspect, pH, GLP-1 (7-36) amide content and purity over time up to 6 weeks at two storage conditions 5° C. and 25° C./60% RH.

| | | t = 2 w | | t = 4 w | | t = 6 w | |
|---|---|---|---|---|---|---|---|
| | | | 25° C./ | | 25° C./ | | 25° C./ |
| Parameters | t0 | 5° C. | 60% RH | 5° C. | 60% RH | 5° C. | 60% RH |
| Visual aspect | Solution | Solution | Solution | Solution | Solution | Solution | Solution |
| pH | 4.4 | 4.3 | 4.3 | 4.3 | 4.3 | 4.3 | 4.3 |
| GLP-1 (7-36) | 0.21 | 0.21 | 0.20 | 0.21 | 0.19 | 0.20 | 0.19 |
| amide content (%) | | | | | | | |
| Purity (%) | 98.8 | 98.6 | 96.5 | 98.0 | 94.7 | 97.9 | 92.6 |

### Example 4. Preparation of a Ready to use Aqueous Solution of Aspartic Acid with GLP-1 (7-36) Amide at a Concentration of 24 mg/mL and Benzalkonium Chloride as Preservative

An ophthalmic aqueous composition at 24 mg/ml of synthetic human glucagon — like peptide (7-36) amide was manufactured at a 5 mL scale.

| GLP-1 (7-36) amide base concentration | 24 mg/mL |
|---|---|
| Component | % (w/w) |
| GLP-1 (7-36) amide acetate salt | 2.69* |
| [GLP-1 (7-36) amide base*] | [2.40] |
| Aspartic acid | 0.19 |
| Polyvinylpyrrolidone | 1.00 |
| Benzalkonium chloride | 0.02 |
| Water for irrigation | 96.10 |

*Taking into account a Batch 1065094 peptide content in the acetate salt of 89.3%. Supplier of the peptide: Hemmo Pharmaceuticals Pvt. Ltd

193.6 mg of aspartic acid were weighted and adjusted to a volume of 100 mL with water for irrigation in a ready to use 250 mL sterile container. The mixture was stirred until complete dissolution.

99 g of the previous solution were sampled in a new reactor equipped with magnetic stirring. 1 g of polyvinylpyrrolidone K90, as viscosifying agent, was weighed and added stepwise into the reactor to the 99 g of the solution previously prepared for 30 minutes to ensure good dissolution. Afterwards, 20.0 mg of benzalkonium chloride, were added and stirred until complete dissolution.

4.87 g of the previous solution were weighted in a reactor equipped with magnetic stirring, 134.5 mg of GLP-1 (7-36) amide as acetate (batch: 1065094, pure peptide content #89.3%) were weighted and added in the previous weighted solution and stirred to get a complete dissolution. The pH of the final solution was 4.5.

The final solution was filtered through syringe filter with polytetrafluoroethylene (PTFE) membrane with 0.2 pm pore size. Type I glass vials (2 mL) were filled with 2 mL o of the previously filtered solution. The vials were closed using 13 mm bromobutyl rubber and sealed with 13 mm aluminium cap.

The ophthalmic solution obtained was then characterized. The solution had a clear solution aspect. Moreover, pH, osmolality and GLP-1 (7-36) amide content and purity by RP-HPLC were determined over time up to 6 weeks at two storage conditions 5° C. and 25° C./60% RH. The RP-HPLC method used is described in Example-1.

Table below shows the visual aspect, pH, osmolality, GLP-1 (7-36) amide content and purity over time up to 12 months at 5° C. and 25° C. 60% HR.

| | | t = 3 M | | t = 6 M | | t = 12 M | |
|---|---|---|---|---|---|---|---|
| | | | 25° C./ | | 25° C./ | | 25° C./ |
| Parameters | t0 | 5° C. | 60% RH | 5° C. | 60% RH | 5° C. | 60% RH |
| pH | 4.5 | 4.5 | 4.6 | 4.6 | ND | ND | ND |
| Osmolality | 55.5 | 57 | 52 | 53 | ND | ND | ND |
| (mOsm/Kg) | | | | | | | |
| GLP-1 (7-36) | 2.35 | 2.24 | 2.05 | 2.17 | 1.89 | 2.18 | 1.47 |
| amide content (%) | | | | | | | |
| Purity (%) | 99.2 | 97.4 | 92.1 | 96.4 | 88.6 | 94.2 | 85.1 |

### Example 5. Preparation of an Ophthalmic Solution Aspartic Acid at pH 4.5 with GLP-1 (7-36) Amide at a Concentration of 2 mg/mL

Step1: Preparation of Freeze-Dried Product Containing GLP-1 (7-36) Amide at a Dose of 4 mg/vial and Aspartic Acid

Freeze-dried product

| Component | Content per vial (mg) |
|---|---|
| GLP-1 (7-36) from acetate salt | 4 |
| (Hemmo Pharmaceuticals Pvt. Ltd) | |
| Aspartic acid | 0.32 |
| WATER FOR IRRIGATION | Traces |
| Nitrogen | Head space |

40.25 mg of aspartic acid were weighted in a ready to use 250 mL sterile container and then adjusted to a volume of 100 mL with water for irrigation. The mixture was stirred until complete dissolution.

59.67 g of the previous solution were weighted in a reactor equipped with magnetic stirring, 334 mg of GLP-1 (7-36) amide as acetate salt (batch: 020217, pure peptide content p90.78%) were weighted and added under stirring until complete dissolution. The pH of the final solution was 4.4.

The final solution consisting on GLP-1 (7-36) amide at a concentration of 5 mg/mL was filtered through polyvinylidene difluoride (PVDF) filter membrane with 0.22 μm pore size.

Prior to lyophilization, 2 mL type-I glass vials intended for lyophilization were filled with 0.8 mL of the solution to obtain a freeze-dried product with a dose of 4 mg/vial of GLP-1 (7-36) amide after lyophilization.

The lyophilized vials were stoppered inside the freeze-drier under N₂ at 500 mbar using a 13 mm bromobutyl rubber and sealed with 13 mm aluminium cap. 67 freeze-dried vials were obtained reaching a final yield around 89%

The freeze-dried vials were then characterized by means of their visual aspect and GLP-1 (7-36) amide content and purity by RP-HPLC over time up to 12 months at three storage conditions 5° C., 25° C./60% RH and 40° C./75% RH. The RP-HPLC method used is described in Example 1.

The obtained cake showed a good intact aspect. Table below shows GLP-1 (7-36) amide content and purity over time up to 12 months at three storage conditions 5° C., 25° C./60% RH and 40° C./75% RH.

| | | t = 2 w | | | t = 1 M | | | t = 12 M | | |
|---|---|---|---|---|---|---|---|---|---|---|
| | | | 25° C. | 40° C. | | 25° C. | 40° C. | | 25° C. | 40° C. |
| Parameters | t0 | 5° C. | 60% RH | 75% RH | 5° C. | 60% RH | 75% RH | 5° C. | 60% RH | 75% RH |
| GLP-1 (7-36) | 3.81 | ND | 3.68 | 3.81 | 3.84 | 3.80 | 3.78 | ND | 3.75 | 3.48 |
| amide content (mg) | | | | | | | | | | |
| Purity (%) | 98.6 | ND | 98.3 | 97.9 | 98.5 | 97.8 | 97.6 | ND | 96.9 | 90.6 |

Step 2: Preparation of an Aqueous Reconstitution Vehicle Consisting on Aqueous Solution with a 1% Polyvinylpyrrolidone K90 as Viscosifyinq Agent

Aqueous vehicle composition

| Component | % (w/w) |
|---|---|
| Polyvinylpyrrolidone | 1.00 |
| Water for irrigation | 99.00 |

2 g of polyvinylpyrrolidone K90, as viscosifying agent, were weighed and added stepwise into the reactor with 198 g of pre-weighted water for irrigation under stirring. The mixture was stirred for 30 minutes to ensure good dissolution. The solution was filtered through polyvinylidene difluoride (PVDF) filter membrane with 0.22 μm pore size.

The reconstitution vehicle solution was then characterized by means of its visual aspect showing a clear solution aspect.

Step 3. Preparation of Reconstituted Product at a Concentration of 2 mg/mL of GLP-1 (7-36) Amide with Aspartic Acid

A total of 27 vials from the Step 1 Example 5 were reconstituted with 2 mL from the reconstitution vehicle from the Step 2 Example 5 to obtain an ophthalmic solution at a GLP-1 (7-36) amide concentration of 2 mg/mL with the composition described below:

| GLP-1 (7-36) amide base concentration | 2 mg/mL |
|---|---|
| Component | % (w/w) |
| GLP-1 (7-36) amide acetate salt | 0.22* |
| [GLP-1 (7-36) amide base*] | [0.20] |
| Aspartic acid | 0.016 |
| Polyvinylpyrrolidone | 1.00 |
| Water for irrigation | 98.76 |

*Taking into account a Batch 020217 peptide content in the acetate salt of 90.78%.

The ophthalmic solution obtained was then characterized. The solution had a clear solution aspect. Moreover, pH (Metrohm 780) and GLP-1 (7-36) amide content and purity by RP-HPLC were determined over time up to 6 weeks at two storage conditions 5° C. and 25° C./60% RH. The RP-HPLC method used is described in Example-1.

Table below shows the visual aspect and pH over time up to 6 weeks at two storage conditions 5° C. and

| | | t = 2 w | | t = 4 w | | t = 6 w | |
|---|---|---|---|---|---|---|---|
| | | | 25° C./ | | 25° C./ | | 25° C./ |
| Parameters | t0 | 5° C. | 60% RH | 5° C. | 60% RH | 5° C. | 60% RH |
| Visual aspect | Solution | Solution | Solution | Solution | Solution | Solution | Solution |
| pH | 4.5 | 4.5 | 4.6 | 4.5 | 4.6 | 4.5 | 4.6 |

Table below shows GLP-1 (7-36) amide content and purity over time up to 6 weeks at three storage conditions 5° C. and 25° C./60% RH.

| | | t = 2 w | | t = 4 w | | t = 6 w | |
|---|---|---|---|---|---|---|---|
| | | | 25° C. | | 25° C. | | 25° C. |
| Parameters | t0 | 5° C. | 60% RH | 5° C. | 60% RH | 5° C. | 60% RH |
| GLP-1 (7-36) | 0.19 | 0.19 | 0.18 | 0.19 | 0.18 | 0.19 | 0.17 |
| amide content (%) | | | | | | | |
| Purity (%) | 99.0 | 98.5 | 96.3 | 98.1 | 93.8 | 97.7 | 92.1 |

### Example 6. Preparation of an Ophthalmic Solution of Acetic/Acetate buffer at pH 4.5 with GLP-1 (7-36) Amide at a Concentration of 2 mg/mL

Step 1: Preparation of a Freeze-Dried Product Containing GLP-1 (7-36) Amide at a Dose of 4 mg/Vial and Mannitol

Freeze-dried product

| Component | Content per vial (mg) |
|---|---|
| GLP-1 (7-36) amide | 4 |
| (Hemmo Pharmaceuticals Pvt. Ltd) | |
| Mannitol | 8 |
| WATER FOR IRRIGATION | Traces |
| Nitrogen | Head space |

445 mg of GLP-1 (7-36) amide acetate (batch: 020217, pure peptide content #90.78%), 800 mg of mannitol, and 78.8 g of water for irrigation were weighted in a ready to use 250 mL sterile container. The mixture was stirred until complete dissolution. The pH of the final solution was 4.9.

The final solution consisting on GLP-1 (7-36) amide at a concentration of 5 mg/mL was filtered through polyvinylidene difluoride (PVDF) filter membrane with 0.22 μm pore size.

Prior to lyophilization, 2 mL type-I glass vials intended for lyophilization were filled with 0.8 mL of the solution to obtain a freeze-dried product with a dose of 4 mg/vial of GLP-1 (7-36) amide after lyophilization.

The lyophilized vials were stoppered inside the freeze-drier under N₂ at 500 mbar using a 13 mm bromobutyl rubber and sealed with 13 mm aluminium cap. 92 freeze-dried vials were obtained reaching a final yield around 92%

The freeze-dried vials were then characterized by means of their visual aspect and GLP-1 (7-36) amide content and purity by RP-HPLC over time up to 6 weeks at three storage conditions 5° C., 25° C./60% RH and 40° C./75% RH. The RP-HPLC method used is described in Example-1.

The obtained cake showed a good aspect. Table below shows GLP-1 (7-36) amide content and purity over time up to 6 weeks at three storage conditions 5° C., 25° C./60% RH and 40° C./75% RH.

| | | t = 2 w | | | t = 4 w | | | t = 6 w | | |
|---|---|---|---|---|---|---|---|---|---|---|
| | | | 25° C. | 40° C. | | 25° C. | 40° C. | | 25° C. | 40° C. |
| Parameters | t0 | 5° C. | 60% RH | 75% RH | 5° C. | 60% RH | 75% RH | 5° C. | 60% RH | 75% RH |
| GLP-1 (7-36) | 4.00 | ND | 4.04 | 4.02 | 4.02 | 4.02 | 3.98 | 4.09 | 3.99 | 4.03 |
| amide content mg | | | | | | | | | | |
| Purity (%) | 98.4 | ND | 98.6 | 97.8 | 98.7 | 98.3 | 97.8 | 98.3 | 97.9 | 97.8 |

Step 2: Preparation of an Aqueous Reconstitution Vehicle Consisting on Acetic/Acetate Buffer Solution (pH 4.4) with a 1% polyvinylpyrrolidone K90 as Viscosifying Agent

Aqueous vehicle composition

| Component | % (w/w) |
|---|---|
| Sodium acetate | 0.13 |
| Ammonium acetate | 0.08 |
| Acetic acid | 0.25 |
| Polyvinylpyrrolidone | 1.00 |
| Water for irrigation | 98.54 |

272 mg of sodium acetate and 154 mg of ammonium acetate were weighted in a ready to use 250 mL sterile container and then adjusted to a volume of 200 mL with water for irrigation. The mixture was stirred until complete dissolution. 0.50 mL of acetic acid was then added to the solution. The pH of the acetic/acetate solution is 4.4.

198 g of the previous solution were sampled in a new reactor equipped with magnetic stirring. 2 g of polyvinylpyrrolidone K90, as viscosifying agent, were weighed and added stepwise into the reactor under stirring for 30 minutes to ensure good dissolution. The solution was filtered through polyvinylidene difluoride (PVDF) filter membrane with 0.22 μm pore size.

The reconstitution vehicle solution was then characterized by means of its visual aspect showing a clear solution aspect.

Step 3 Preparation of Reconstituted Product at a Concentration of 2 mg/mL of GLP-1 (7-36) Amide with Acetic/Acetate Buffer

A total of 26 vials form the Step 1 Example 6 were reconstituted with 2 mL from the vehicle form the Step 2 Example 6 to reach an ophthalmic solution at a GLP-1 (7-36) amide concentration of 2 mg/mL with the composition described below:

| GLP-1 (7-36) amide concentration | 2 mg/mL |
|---|---|
| Component | % (w/w) |
| GLP-1 (7-36) amide acetate salt | 0.22* |
| [GLP-1 (7-36) amide base*] | [0.20] |
| Sodium acetate | 0.13 |
| Ammonium acetate | 0.08 |
| Acetic acid | 0.25 |
| Mannitol | 0.40 |
| Polyvinylpyrrolidone | 1.00 |
| WATER FOR IRRIGATION | 97.92 |

*Taking into account a peptide content in the acetate salt of 90%

The ophthalmic solution obtained was then characterized. The solution had a clear solution aspect. Moreover, pH and GLP-1 (7-36) amide content and purity by RP-HPLC were determined over time up to 6 weeks at two storage conditions 5° C. and 25° C./60% RH. The RP-HPLC method used is described in Example-1.

Table below shows the visual aspect and pH over time up to 6 weeks at two storage conditions 5° C. and 25° C./60% RH.

| | | t = 2 w | | t = 4 w | | t = 6 w | |
|---|---|---|---|---|---|---|---|
| | | | 25° C./ | | 25° C./ | | 25° C./ |
| Parameters | t0 | 5° C. | 60% RH | 5° C. | 60% RH | 5° C. | 60% RH |
| Visual aspect | Solution | Solution | Solution | Solution | Solution | Solution | Solution |
| pH | 4.6 | 4.5 | 4.5 | 4.5 | 4.5 | 4.5 | 4.5 |

Table below shows GLP-1 (7-36) amide content and purity over time up to 6 weeks at three storage conditions 5° C., 25° C./60% RH and 40° C./75% RH.

| | | t = 2 w | | t = 4 w | | t = 6 w | |
|---|---|---|---|---|---|---|---|
| | | | 25° C. | | 25° C. | | 25° C. |
| Parameters | t0 | 5° C. | 60% RH | 5° C. | 60% RH | 5° C. | 60% RH |
| GLP-1 (7-36) | 0.20 | 0.20 | 0.18 | 0.20 | 0.17 | 0.20 | 0.16 |
| amide content (%) | | | | | | | |
| Purity (%) | 99.0 | 97.6 | 92.3 | 96.9 | 88.0 | 95.8 | 87.7 |

### Example 7. Preparation of an Ophthalmic Solution of Aspartic Aacid at pH 4.4 with GLP-1 (7-36) Amide at a Concentration of 2 mg/mL

Step 1 from Example 6: Preparation of a freeze-dried product containing GLP-1 (7-36) amide at a dose of 4 mg/vial and mannitol

Step 2: Preparation of an Aqueous Reconstitution Vehicle Consisting on Aqueous Solution of Aspartic Acid with a 1% polyvinylpyrrolidone K90 as Viscosifying Agent

Aqueous vehicle composition

| Component | % (w/w) |
|---|---|
| Aspartic acid | 0.016 |
| Polyvinylpyrrolidone | 1.00 |
| Water for irrigation | 98.98 |

32.2 mg of aspartic acid were weighted in a ready to use 250 mL sterile container and then poured into 200 mL of water for irrigation. The mixture was stirred until complete dissolution.

198 g of the previous solution were sampled in a new reactor equipped with magnetic stirring. 2 g of polyvinylpyrrolidone K90, as viscosifying agent, were weighed and added stepwise into the reactor under stirring for 30 minutes to ensure good dissolution. The solution was filtered through polyvinylidene difluoride (PVDF) filter membrane with 0.22 μm pore size.

The reconstitution vehicle solution was then characterized by means of its visual aspect showing a clear solution aspect.

Step 3: Preparation of Reconstituted Product at a Concentration of 2 mg/mL of GLP-1 (7-36) Amide with Mannitol and Aspartic Acid

A total of 25 vials form the from the Step 1 Example 6 were reconstituted with 2 mL from the vehicle form the from the Step 2 Example 7 to reach an ophthalmic solution at a GLP-1 (7-36) amide concentration of 2 mg/mL with the composition described below:

| GLP-1 (7-36) amide concentration | 2 mg/mL |
|---|---|
| Component | % (w/w) |
| GLP-1 (7-36) amide acetate salt | 0.22* |
| [GLP-1 (7-36) amide base*] | [0.20] |
| Aspartic acid | 0.016 |
| Mannitol | 0.40 |
| Polyvinylpyrrolidone | 1.00 |
| WATER FOR IRRIGATION | 98.36 |

*Taking into account a Batch 020217 peptide content in the acetate salt of 90.78%

The ophthalmic solution obtained was then characterized. The solution had a clear solution aspect. Moreover, pH and GLP-1 (7-36) amide content and purity by RP-HPLC were determined over time up to 6 weeks at two storage conditions 5° C. and 25° C./60% RH. The RP-HPLC method used is described in Example-1.

Table below shows the visual aspect and pH over time up to 6 weeks at two storage conditions 5° C. and 25° C./60% RH.

| | | t = 2 w | | t = 4 w | | t = 6 w | |
|---|---|---|---|---|---|---|---|
| | | | 25° C./ | | 25° C./ | | 25° C./ |
| Parameters | t0 | 5° C. | 60% RH | 5° C. | 60% RH | 5° C. | 60% RH |
| Visual aspect | Solution | Solution | Solution | Solution | Solution | Solution | Solution |
| pH | 4.4 | 4.4 | 4.4 | 4.4 | 4.5 | 4.5 | 4.5 |

Table below shows GLP-1 (7-36) amide content and purity over time up to 6 weeks at two storage conditions 5° C. and 25° C./60% RH.

| | | t = 2 w | | t = 4 w | | t = 6 w | |
|---|---|---|---|---|---|---|---|
| | | | 25° C. | | 25° C. | | 25° C. |
| Parameters | t0 | 5° C. | 60% RH | 5° C. | 60% RH | 5° C. | 60% RH |
| GLP-1 (7-36) | 0.20 | 0.21 | 0.19 | 0.20 | 0.17 | 0.20 | 0.16 |
| amide content (%) | | | | | | | |
| Purity (%) | 98.8 | 97.9 | 93.6 | 97.4 | 90.4 | 96.6 | 87.9 |

### Example 8. Preparation of an Ophthalmic Solution of Aspartic Acid at pH 4.5 with GLP-1 (7-36) Amide at a Concentration of 2 mg/mL

Step 1: Preparation of Freeze-Dried Product Containing GLP-1 (7-36) Amide at a Dose of 10 mg/Vial and Aspartic Acid

Freeze-dried product

| Component | Content per vial (mg) |
|---|---|
| GLP-1 (7-36) from acetate salt | 10 |
| (Hemmo Pharmaceuticals Pvt. Ltd) | |
| Aspartic acid | 0.8 |
| WATER FOR IRRIGATION | Traces |
| Nitrogen | Head space |

480.0 mg of aspartic acid were weighted and poured in a 1 L reactor equipped with magnetic stirring containing 500 mL of water for irrigation. The mixture was stirred until complete dissolution.

6.72 g of GLP-1 (7-36) amide as acetate salt (pure peptide content #89.3%) were weighted and added under stirring until complete dissolution. The pH of the final solution was 4.5. This solution was finally brought up to a final volume of 900 mL with water for irrigation

The final solution consisting on GLP-1 (7-36) amide at a concentration of 6.67 mg/mL was filtered through polyvinylidene difluoride (PVDF) filter membrane with 0.22 μm pore size.

Prior to lyophilization, 6 mL type-I glass vials intended for lyophilization were filled with 1.5 mL of the solution to obtain a freeze-dried product with a dose of 10 mg/vial of GLP-1 (7-36) amide after lyophilization.

The lyophilized vials were stoppered inside the freeze-drier under N₂ at 500 mbar using a 18 mm bromobutyl rubber and sealed with 18 mm aluminium cap. 495 freeze-dried vials were obtained reaching a final yield around 93.4%

The freeze-dried vials were then characterized by means of their visual aspect and GLP-1 (7-36) amide content and purity by RP-HPLC over time up to 3 months at two storage conditions 5° C. and 25° C./60% RH. The RP-HPLC method used is described in Example 1.

The obtained cake showed a good intact aspect. Table below shows purity over time up to 18 months at two storage conditions 5° C. and 25° C./60% RH.

| | | t = 1 month | | t = 3 months | | t = 18 months | |
|---|---|---|---|---|---|---|---|
| | | | 25° C./ | | 25° C./ | | 25° C./ |
| Parameters | t0 | 5° C. | 60% RH | 5° C. | 60% RH | 5° C. | 60% RH |
| Purity (%) | 106.3 | 103.1 | 104.3 | 102.2 | 102.1 | 101.2 | 99.3 |

Step 2: Preparation of an Aqueous Reconstitution Vehicle Consisting on Aqueous Solution with a 1% polyvinylpyrrolidone K90 as Viscosifying Agent

Aqueous vehicle composition

| Component | % (w/w) |
|---|---|
| Polyvinylpyrrolidone | 1.00 |
| Benzalkonium chloride | 0.025 |
| Water for irrigation | 98.98 |

50 g of polyvinylpyrrolidone K90, as viscosifying agent, were weighed and added stepwise into the reactor with 3 L of pre-weighted water for irrigation under stirring. The mixture was stirred for 1 hour to ensure good dissolution.

1.25 g of benzalkonium chloride were weighed and added to the mixture obtained above until complete dissolution. The above solution was brought up to a final volume of 5 L and then it was filtered through polyvinylidene difluoride (PVDF) filter membrane with 0.22 μm pore size.

The reconstitution vehicle solution was then characterized by means of its visual aspect showing a clear solution aspect.

Step 3. Preparation of Reconstituted Product at a Concentration of 2 mg/mL of GLP-1 (7-36) Amide with Aspartic Acid

A total of 152 vials from the Step 1 Example 8 were reconstituted with 5 mL from the reconstitution vehicle from the Step 2 Example 8 to obtain an ophthalmic solution at a GLP-1 (7-36) amide concentration of 2 mg/mL with the composition described below:

| GLP-1 (7-36) amide base concentration | 2 mg/mL |
|---|---|
| Component | % (w/w) |
| GLP-1 (7-36) amide acetate salt | 0.22* |
| [GLP-1 (7-36) amide base*] | [0.20] |
| Aspartic acid | 0.016 |
| Benzalkonium chloride | 0.025 |
| Polyvinylpyrrolidone | 1.00 |
| Water for irrigation | 98.76 |

*Taking into account a Batch 020217 peptide content in the acetate salt of 90.78%.

The ophthalmic solution obtained was then characterized. The solution had a clear solution aspect with a viscosity at 20° C. of 3.8 cSt. Moreover, pH and purity by RP-HPLC were determined over time up to 3 months at two storage conditions 5° C. and 25° C./60% RH. The RP-HPLC method used is described in Example-1.

Table below shows the visual aspect and pH over time up to 6 months at two storage conditions 5° C. and 25° C./60% RH.

| | | t = 1 month | | t = 3 months | | t = 6 months | |
|---|---|---|---|---|---|---|---|
| | | | 25° C./ | | 25° C./ | | 25° C./ |
| Parameters | t0 | 5° C. | 60% RH | 5° C. | 60% RH | 5° C. | 60% RH |
| Visual aspect | Solution | Solution | Solution | Solution | Solution | Solution | Solution |
| pH | 4.6 | 4.7 | 4.7 | 4.7 | 4.7 | 4.7 | 4.7 |

Table below shows purity over time up to 3 months at two storage conditions 5° C. and 25° C./60% RH.

| | | t = 1 month | | t = 3 months | | t = 6 months | |
|---|---|---|---|---|---|---|---|
| | | | 25° C./ | | 25° C./ | | 25° C./ |
| Parameters | t0 | 5° C. | 60% RH | 5° C. | 60% RH | 5° C. | 60% RH |
| Purity (%) | 103.4 | 103.4 | 96.2 | 102.5 | 91.0 | 101.0 | 87.1 |

### Example 9. Preparation of an Ophthalmic Solution of Glutamic Acid with GLP-1 (7-36) Amide at a Concentration of 2 mg/mL Ready to Use

| GLP-1 (7-36) amide base concentration | 2 mg/mL |
|---|---|
| Component | % (w/w) |
| GLP-1 (7-36) amide acetate salt | 0.23* |
| [GLP-1 (7-36) amide base*] | [0.21] |
| Glutamic acid | 0.018 |
| Water for irrigation | 99.75 |

*Taking into account a Batch 020217 peptide content in the acetate salt of 90.78%. Supplied by Hemmo Pharmaceuticals Pvt. Ltd.

1.80 mg of glutamic acid were weighted and then poured into 10 mL of water for irrigation in a ready to use 15 mL sterile container. The mixture was stirred until complete dissolution.

11.45 mg of GLP-1 (7-36) amide in an acetate salt (pure peptide content p90.78%) were weighted in a 6 mL vial.

5 mL of the previous glutamic acid solution was added in the 6 mL vial containing the GLP-1 (7-36) amide in an acetate salt and stirred to get a complete dissolution. The pH of the final solution was 4.6.

### Example 10. Preparation of an Ophthalmic Solution of Glutamic Acid with GLP-1 (7-36) Amide at a Concentration of 2 mg/mL Ready to Use

| GLP-1 (7-36) amide base concentration | 2 mg/mL |
|---|---|
| Component | % (w/w) |
| GLP-1 (7-36) amide acetate salt | 0.22* |
| [GLP-1 (7-36) amide base*] | [0.20] |
| Glutamic acid | 0.018 |
| Polyvinylpyrrolidone | 1.00 |
| Water for irrigation | 98.76 |

*Taking into account a Batch 020217 peptide content in the acetate salt of 90.78%. Supplied by Hemmo Pharmaceuticals Pvt. Ltd.

1.80 mg of glutamic acid were weighted and then poured into 10 mL of water for irrigation in a ready to use 15 mL sterile container. The mixture was stirred until complete dissolution.

9.9 g of the previous solution were sampled in a new reactor equipped with magnetic stirring. 0.1 g of polyvinylpyrrolidone K90, as viscosifying agent, were weighed and added stepwise into the reactor to the 9.9 g of the solution previously prepared for 30 minutes to ensure good dissolution.

10.0 g of the previous solution were weighted in a reactor equipped with magnetic stirring, 22.3 mg of GLP-1 (7-36) amide as acetate salt (batch: 020217, pure peptide content #90.78%) were weighted and added in the previous solution and stirred to get a complete dissolution. The pH of the final solution was 4.6.

### REFERENCES CITED IN THE APPLICATION

Schmidt et al., “Neurodegenerative Diseases of the Retina and Potential for the Protection and Recovery”, Current Neuropharmacology, 2008, Vol. No. 6, pp. 164-178.

Simó et al., “Neurodegeneration is an early event in diabetic retinopathy: therapeutic implications”, Br. J. Ophthalmol., 2012, vol. 96, pp. 1285-1290

WO2007062434

Altschul et al., “Basic local alignment search tool”, 1990, J. Mol. Biol, v. 215, pages 403-410

1. An ophthalmic pharmaceutical composition comprising:
a peptide or a pharmaceutically acceptable salt or solvate thereof with a sequence length from 13 to 50 amino acids, the N-terminal region of said peptide consisting of the sequence:
HXaa¹EGTFTSDXaa²SXaa³Xaa⁴(SEQ ID NO: 1) wherein:
Xaa¹ is an amino acid selected from alanine and glycine;
Xaa² is an amino acid selected from valine and leucine;
Xaa³ is an amino acid selected from serine and lysine;
Xaa⁴ is an amino acid selected from tyrosine and glutamine; and
histidine is the N-terminal residue; and
one or more pharmaceutically acceptable excipients or carriers;
wherein the pH value of the composition is between 4.0 and 4.8 and the osmolality ranges between 0.5 and 200 mOsm/kg.

2. The ophthalmic pharmaceutical composition according to claim 1, wherein the sequence length is from 30 to 40 amino acids.

3. The ophthalmic pharmaceutical composition according to claim 1, wherein Xaa¹ is alanine, Xaa² is valine, Xaa³ is serine, and Xaa⁴ is tyrosine.

4. The ophthalmic pharmaceutical composition according to claim 1, wherein the peptide is a mammal glucagon-like peptide-1, or a pharmaceutically acceptable salt thereof.

5. The ophthalmic pharmaceutical composition according to claim 1, wherein the peptide is:
(a) a peptide comprising or consisting of the amino acid sequence SEQ ID NO: 2 or a pharmaceutically acceptable salt thereof; or, alternatively,
(b) a peptide with an amino acid sequence having at least 85% of identity degree with SEQ ID NO: 2 or a pharmaceutically acceptable salt thereof, which contains said N-terminal region or, alternatively,
(c) a peptide with a sequence length up to 50 amino acids comprising an amino acid sequence having at least 85% of identity degree with SEQ ID NO: 2 or a pharmaceutically acceptable salt thereof, which contains said N-terminal region; or, alternatively,
(d) a fragment of a peptide having at least 85% of identity degree with SEQ ID NO: 2 or a pharmaceutically acceptable salt thereof, provided that the fragment has an amino acid length from 14 to 49 amino acids and includes the N-terminal region.

6. The ophthalmic pharmaceutical composition according to claim 1, wherein the peptide is a pharmaceutically acceptable salt of the sequence SEQ ID NO: 2 or the peptide is the sequence SEQ ID NO: 2.

7. The ophthalmic pharmaceutical composition according to claim 1, wherein the pH value is between 4.1 and 4.8.

8. The ophthalmic pharmaceutical composition according to claim 1, wherein the osmolality ranges from 1 to 150 mOsm/kg.

9. The ophthalmic pharmaceutical composition according to claim 1, wherein at least one of the one or more pharmaceutically acceptable excipients or carriers is selected from the group of a stabilizing agent, a viscosifying agent, a buffering agent and mixtures thereof.

10. The ophthalmic pharmaceutical composition according to claim 9, wherein the stabilizing agent is aspartic acid or glutamic acid.

11. The ophthalmic pharmaceutical composition according to claim 1 further comprising an effective amount of a preservative.

12. The ophthalmic pharmaceutical composition according to claim 1, wherein the preservative is selected from edetate sodium, benzalkonium chloride, centrimonium chloride, sodium perborate, stabilized oxychloro complex, sorbic acid, thimerosal, polyquarternium-1, polyhexamethylene biguanide, chlorobutanol, phenylethyl alcohol, methylparaben, propylparaben, a combination of boric acid, sorbic acid, and propylene glycol; and mixtures thereof.

13. A lyophilizate comprising the peptide as defined in claim 1 or a pharmaceutically acceptable salt thereof, and a pharmaceutically acceptable amount of a stabilizing agent and/or buffering agent wherein said lyophilizate is suitable for preparing the ophthalmic pharmaceutical composition by reconstitution.

14. A process for preparing the ophthalmic pharmaceutical composition according to claim 1, which comprises the step of reconstituting the lyophilizate as defined in claim 13 a lyophilizate comprising the peptide or a pharmaceutically acceptable salt thereof, and a pharmaceutically acceptable amount of a stabilizing agent and/or buffering agent, with an aqueous vehicle composition comprising one or more pharmaceutically acceptable carriers or excipients, particularly an aqueous vehicle composition comprising at least one viscosifying agent and optionally at least one preservative.

15. An ophthalmic pharmaceutical composition according to claim 1 for use in the topical eye treatment and/or prevention of a retinal neurodegenerative disease.

16. A kit comprising the lyophilizate as defined in claim 13 and a physiologically acceptable vehicle composition comprising one or more pharmaceutically acceptable excipients or carriers, for reconstituting the peptide.

17. A kit comprising the ophthalmic pharmaceutical composition as defined in claim 1, a container for holding the pharmaceutical composition and a drop dispenser adapted for administering a volume of the composition.

18. The ophthalmic pharmaceutical composition according to claim 1, wherein the peptide is an acetate salt of the sequence SEQ ID NO: 2.

19. The ophthalmic pharmaceutical composition according to claim 12, wherein the preservative is benzalkonium chloride.

20. The process for preparing the ophthalmic pharmaceutical composition according to claim 14, wherein the aqueous vehicle composition comprising one or more pharmaceutically acceptable carriers or excipients is an aqueous vehicle composition comprising at least one viscosifying agent and optionally at least one preservative
